(19) United States
(12) Patent Application Publication
(10) Pub. No.: US 2020/0345898 A1
(43) Pub. Date: Nov. 5, 2020

(54) EXTRACELLULAR MATRIX AND ITS USE FOR REGULATING THE DIFFERENTIATION OF MESENCHYMAL STEM CELLS

(71) Applicant: CONSEJO SUPERIOR DE INVESTIGACIONES CIENTÍFICAS (CSIC), MADRID (ES)

(72) Inventors: Fernando RODRÍGUEZ PASCUAL, MADRID (ES); Tamara ROSELL GARCÍA, MADRID (ES)

(21) Appl. No.: 16/955,441

(22) PCT Filed: Dec. 18, 2018
(86) PCT No.: PCT/EP2018/085634

§ 371 Date: Jun. 18, 2020

(30) Foreign Application Priority Data
Dec. 20, 2017 (ES) P 201731441

(51) Int. Cl. A61L 27/54; A61L 27/36; A61K 38/18; A61K 38/44; C12N 5/0775

(52) U.S. Cl. CPC A61L 27/54; A61L 27/3633; A61K 38/1875; A61K 38/44; C12N 5/0662; C12N 2501/155; A61L 2430/00; C12N 2533/90; C12N 2533/54; C12N 2501/71; C12Y 104/03013

(57) ABSTRACT

The present invention relates to an extracellular matrix comprising ECM producer cells, a lysyloxidase (LOX), and bone morphogenetic protein-1 (BMP1), and its use for regulating the differentiation of mesenchymal stem cells and increasing the synthesis and/or deposit of collagen in an extracellular matrix. The present invention also relates to a method for obtaining said extracellular matrix comprising incubating cells in the presence of a composition comprising a lysyl oxidase, or a fragment thereof, and bone morphogenetic protein-1, or a fragment thereof.

The invention relates to an extracellular matrix (ECM) with an increased deposit of collagen comprising a lysyl oxidase (LOX) and bone morphogenetic protein-1 (BMP1), and its use for regulating the differentiation of mesenchymal stem cells. The invention also discloses an in vitro method for obtaining said extracellular matrix. Thus, the present invention relates to cell culture methodology, more specifically, to enhance formation of extracellular matrices.

### STATE OF ART

Tissue engineering is emerging as a powerful therapeutic strategy to treat injured or degenerated tissues by implanting natural, synthetic, or semisynthetic tissue and organ mimics. Cell-derived ECM-based biomaterials exploit the inherent capacity of cells to create highly sophisticated supramolecular assemblies.

The extracellular matrix (ECM) is a dynamic microenvironment that importantly influences a number of cellular processes, including cell proliferation, adhesion, migration, and differentiation, as well as plays key roles in homeostasis and regeneration of tissues and organs. Tissue engineering has exploited these properties and ECM-based biomaterials are today more than a promising therapy for tissue repair and regeneration. The use of native ECM substrates over artificially assembled ECM scaffolds allows for better preservation of the appropriate cell growth microenvironment, thereby speeding up the repair of damaged tissue. Therefore, there remains a strong interest in developing techniques and protocols to enhance the innate capacity of cells to create their own ECM in vitro. However, despite significant progresses, optimal conditions for rapid and efficient deposition of ECM components, mainly collagen, the most important structural biomolecule, are still missing. This behavior has been attributed to the diluted culture media that severely limit extracellular post-translational modifications of collagen, namely the cleavage of the C propeptide, a reaction catalyzed by C-proteinase/bone morphogenetic protein 1 (BMP1), and the formation of covalent cross-links, initiated by members of the lysyl oxidase (LOX) family. These enzymatic activities are intimately intricate as BMP1 also catalyzes the proteolytic activation of the precursor of LOX to yield the active form.

Several approaches have been developed to facilitate collagen deposition in standard cell culture conditions, including ascorbic acid and serum supplementation. One interesting approach was designed based on the addition of inert macromolecules in the culture media in order to imitate a dense extracellular space, a biophysical phenomenon known as macromolecular crowding. Under this principle, the addition of dextran sulfate (DxS) or Ficoll™ has been reported to enhance the capacity of a number of different cell cultures, including fibroblasts, keratinocytes, tenocytes or chondrocytes, to deposit abundant extracellular matrix.

However, standard cell culture conditions are far from ideal given the fact that the diluted microenvironment does not favor the production of ECM components. This is particularly true for collagen, the most important structural biomolecule, as its synthesis and deposition onto matrix is enzymatically rate-limited.

MSCs are attractive candidates for biological cell-based tissue repair approaches because of their extensive proliferative ability in culture while retaining their mesenchymal multilineage differentiation potential. In addition to its undoubted scientific interest, the prospect of monitoring and controlling MSC differentiation is a crucial regulatory and clinical requirement. Hence, the molecular regulation of MSC differentiation has been extensively studied. Most of the studies are in vitro, because the identity of MSCs in their tissues of origin in vivo remains undefined. Building on the information coming from developmental biology studies of embryonic skeletogenesis, several signaling pathways and transcription factors have been investigated and shown to play critical roles in MSC differentiation. In particular, the Wnt and transforming growth factor-β/bone morphogenetic protein signaling pathways are well known to modulate in MSCs the molecular differentiation into cartilage and bone. Relevant to the emerging concept of stem cell niches is the demonstration that physical factors can also participate in the regulation of MSC differentiation. Knowledge of the regulation of MSC differentiation will be critical in the design of three-dimensional culture systems and bioreactors for automated bioprocessing through mathematical models applied to systems biology and network science.

The patent application US2012/178159 concerns materials and methods for growing and expanding mammalian MSC while maintaining their undifferentiated phenotype, self-renewal ability, and/or multi-lineage potential. In one embodiment, a method of the invention comprises i) seeding freshly isolated MSC on a planar surface, such as plastic tissue culture plates, or on a 3-D scaffold and growing the cells under physiological or low O2 tension (e.g., lower than 20% O2) for a period of time sufficient to support formation of 3-D ECM network; ii) decellularizing the cultures on the plates or the 3-D scaffold to obtain decellularized ECM matrices thereon; and iii) reseeding the decellularized matrices on the plates or 3-D scaffold with MSCs, whereby the reseeded MSCs grow on the plate or scaffold that comprises cell-derived 3-D ECM and maintain an undifferentiated phenotype.

The patent application US2007/0269886 discloses a cell culture product for propagating embryonic stem cells, and maintaining their self-renewal and pluripotency characteristics for extended periods of time in culture. The cell culturing product includes a substrate and a coating solution. The coating solution includes a mixture of extracellular matrix proteins and an aqueous solvent, wherein the total protein concentration in the coating solution is about 10 μg/mL to about 1 mg/mL. While this patent application describes a cell culture product including a coating solution functioning as a surrogate of the extracellular matrix, capable of maintaining the undifferentiated phenotype of the stem cells, this extracellular matrix does not display the typical features of a physiological matrix, both at quantitative, i.e. the extent of the matrix components forming such product, and qualitative levels, such as the proper cross-linking of collagen. Therefore, there is a need in the state of the art for providing a more physiological matrix for culturing stem cells while maintaining their undifferentiated phenotype.

### DESCRIPTION OF THE INVENTION

The inventors have discovered that implementing fibroblast cultures with supernatants enriched in LOX and BMP1 from stable HEK293 cell lines strongly increased the deposition of collagen onto the insoluble matrix at the expense of the soluble fraction in the extracellular medium (Example 1). Using decellularization protocols, they have also showed that fibroblast-derived matrices regulate adipogenic and osteogenic differentiation of human mesenchymal stem cells (MSC), and that this effect was modulated by LOX/BMP1 (Example 2). These results support a convenient protocol to enhance the capacity of in vitro cell cultures to deposit collagen in the extracellular matrix, which represents a promising approach for application in tissue engineering, further to provide evidence that fibroblast-derived matrices are able to regulate the adipogenic and osteogenic differentiation of human MSC, a powerful cell tool in regenerative medicine.

Thus, in an aspect, the present invention relates to an extracellular matrix (ECM), hereinafter “ECM of the invention”, comprising a lysyl oxidase (LOX), or a fragment thereof, and a bone morphogenetic protein-1 (BMP1), or a fragment thereof.

The term “extracellular matrix” or “ECM” refers to a collection of extracellular molecules secreted by mammalian tissues cells that provides structural and biochemical support to the surrounding cells, particularly cells of connective tissue, for instance such cells as fibroblasts, osteoblasts, chondrocytes, epithelial cells, smooth muscle cells, adipocytes, and mesenchymal cells, and which material in vivo surrounds and supports those cells. Typically, the ECM is composed of fibres embedded in what is commonly referred to as ‘ground substance’. The fibers are composed of structural proteins, generally collagen and/or elastin. In aspects of the present invention, the fibers of the matrix are preferably collagen. Particularly suitable collagens are fibril-forming collagens. Type I collagen, type II collagen, type III collagen, type IV collagen or type X collagen are particularly preferred. Most preferred is type I collagen. The ‘ground substance’ is composed of proteoglycans (or mucopolysaccharides) and may comprise functionality-providing proteins such as fibrillin, fibronectin, and/or laminin. In aspects of the invention, the ECM suitably comprises at least one proteoglycan as a component of the ground substance. The proteoglycan is composed of a core protein with pending glycosaminoglycan (GAG) molecules. Suitable GAGs are for instance hyaluronic acid, chondroitin-4-sulfate, chondroitin-6-sulphate, dermatan sulphate, heparan sulphate, heparin sulphate, and keratan sulfate. The GAGs are preferably linked to the core protein via a trisaccharide linker (e.g. a GalGalXyl linker). Exemplary proteoglycans are decorin, biglycan, versican and aggrecan. The proteoglycans may optionally be interconnected by hyaluronic acid molecules. Alternatively, multiple proteoglycans may be attached to a single hyaluronic acid backbone. In both cases the ground substance forms a polymer network or gel capable of holding water. The network may further comprise such proteins as: glycoproteins such as laminin, entactin, tenascin, fibrillin or fibronectin, for improving structural integrity of the network and for the attachment of cells to the ECM; osteocalcin (Gla protein), as a protein that binds calcium during mineralization; osteonectin, which serves a bridging function between collagen and mineral component; and sialoproteins, such as bone sialoprotein (BSP), osteopontin (OPN), dentin matrix protein-1 (DMP1), dentin sialophosphoprotein (DSPP) and matrix extracellular phosphoglycoprotein (MEPE). The matrix may further comprise cytokines and growth factors. Suitable cytokines and growth factors include osteoprotegerin (OPG), epidermal growth factor (EGF), fibroblast growth factors (bFGF, FGF-I, and FGF-2), interferon-α (IFN-α), interleukins (IL-I, IL-4, IL-6, IL-IO, and IL-II), platelet-derived growth factor (PDGF), transforming growth factors (TGF-α and TGF-β), tumor necrosis factors (TNFs), insulin-like growth factors (IGF-I and IGF-II), osteoclast differentiation factor (ODF, also known as OPGL [osteoprotegerin ligand], EANKL [receptor activator of NFB ligand], TRANCE [TNF-related activation-induced cytokine]), and macrophage colony-stimulating factor (M-CSF). Most of these (IL-I, IL-4, IL-6, IL-II, TNF, EGF, bFGF, FGF-2, PDGF, and M-CSF) stimulate bone resorption. Some (IGF-I and IGF-II, FGF-2, and TGF-3) enhance bone formation, while others (OPG) inhibit bone resorption. Still others (PDGF and TGF-β) also stimulate proliferation and differentiation of collagen-synthesizing cells.

The surface onto which the ECM of this invention is deposited may be capable of adhering the cells to be cultured and capable of releasing the cells when the culture process is complete. It is well known that animal cells in particular adhere well to surfaces which carry high densities of sodium ions. They therefore adhere to materials which tend to acquire a negative charge and thus bind sodium ions. The surface may be made from a material to which cells adhere or be made up from an inert support and coated with such a material. Suitable materials include plastics, materials such as nylon, polycarbonate, polystyrene, epoxyresins, silicone rubber, cellulose acetate, cellulose nitrate, cellophane, polyethylene terephthalate, polyformaldehyde, fluorinated ethylenepropylene co-polymer, polyphenylene oxide, polypropylene mica, carbon, collagen, insoluble inert metal oxides, phosphates, silicates or carbides, silicon carbide, inert metals such as stainless steel, aluminium, titanium or palladium, or ceramics or glass. It is sometimes necessary to modify the characteristics of the surface by applying a coating of a material less adhesive to cells, which eases their removal. Coating materials that render cells more readily removable from matrix surfaces are polyfluorinated hydrocarbons such as polytetrafluoroethylene, or silicones such as polymethylhydrogensiloxane. On the other hand, a surface of low adhesive capacity may be altered to give better adhesion by the application of a suitable coating. Thus the particular surface coating which is employed will depend upon the type of cells to be cultured and whether harvesting from the matrix is required. A suitable coating or combination of coating for any particular application may be determined empirically. Materials that may be suitable for the formation of matrices for use in this invention include polycarbonate, nylon 6, nylon 11, nylon 12, glass, polyformaldehyde, polypropylene and 2,6-dimethylphenyleneoxide. Coated matrices that may be suitable for the formation of matrices for use in this invention include polycarbonate coated with polytetrafluoroethylene, silicone, polymethylhydrogensiloxane; glass coated with silicone, polytetrafluoroethylene or stearic acid; or polyethyleneterephthalate, nylon 6, nylon 11 and nylon 12, each coated with polytetrafluoroethylene. In any specific application of this method the particular material of choice may be determined by incidental factors such as the method by which the matrix is to be sterilized.

The ECM of the invention comprises a lysyl oxidase (LOX) protein. The LOX protein, also known as protein-lysine 6-oxidase, is a protein that, in humans, is encoded by the LOX gene. In humans, the LOX gene is located on chromosome 5q23.3-31.2. The DNA sequence encodes a polypeptide of 417 amino acids, the first 21 residues of which constitute a signal peptide, with a weight of approximately 32 kDa. The carboxy terminus-end contains the active copper (II) ion, lysine, tyrosine, and cysteine residues that comprise the catalytically active site. While alternative splicing forms have been described to give rise to multiple transcript variants, this invention only refers to the isoform 1 of LOX protein (preprotein, NCBI Reference Sequence NP_002308.2 (SEQ ID NO: 1)), the only one that keeps the processing site by BMP1 protease, and therefore is proteolytically processed and activated by BMP1.

```
SEQ ID NO: 1
    1 MRFAWTVLLL GPLQLCALVH CAPPAAGQQQ PPREPPAAPG AWRQQIQWEN NGQVFSLLSL
  61 GSQYQPQRRR DPGAAVPGAA NASAQQPRTP ILLIRDNRTA AARTRTAGSS GVTAGRPRPT
121 ARHWFQAGYS TSRAREAGAS RAENQTAPGE VPALSNLRPP SRVDGMVGDD PYNPYKYSDD
181 NPYYNYYDTY ERPRPGGRYR PGYGTGYFQY GLPDLVADPY YIQASTYVQK MSMYNLRCAA
241 EENCLASTAY RADVRDYDHR VLLRFPQRVK NQGTSDFLPS RPRYSWEWHS CHQHYHSMDE
301 FSHYDLLDAN TQRRVAEGHK ASFCLEDTSC DYGYHRRFAC TAHTQGLSPG CYDTYGADID
361 CQWIDITDVK PGNYILKVSV NPSYLVPESD YTNNVVRCDI RYTGHHAYAS GCTISPY
```

In a particular embodiment of the ECM of the invention, LOX protein comprises an amino acid sequence with an identity of at least 70, 75, 80, 85, 90, 95, 96, 97, 98 or 99% with SEQ ID NO: 1.

In the present invention, “identity” or “sequence identity” is understood to mean the degree of similarity between two nucleotide or amino acid sequences obtained by aligning the two sequences. Depending on the number of common residues between the aligned sequences, a different degree of identity, expressed as a percentage, will be obtained. The degree of identity between two amino acid sequences may be determined by conventional methods, for example, by standard sequence alignment algorithms known in the state of the art, such as, for example, BLAST [Altschul S. F. et al. Basic local alignment search tool. J Mol Biol. 1990 Oct. 5; 215(3): 403-10]. The BLAST programmes, for example, BLASTN, BLASTX, and T BLASTX, BLASTP and TBLASTN, are in the public domain at The National Center for Biotechonology Information (NCBI) website. The skilled person in the art understands that mutations in the nucleotide sequence of genes that lead to conservative amino acid substitutions at non-critical positions for the functionality of the protein are evolutionarily neutral mutations which do not affect its global structure or its functionality. Thus, variants of the LOX protein of SEQ ID NO: 1 are encompassed within the context of the present invention. Apart from humans, other sources from which LOX protein variants can be obtained include, without limiting to, non-human animals such as non-human primates, pigs, mice and rats among others. In a more particular embodiment, the LOX protein comprises the sequence SEQ ID NO: 1. The present invention also encompasses fragments of the LOX protein, being said fragments considered variants of the LOX protein. As use herein, the term “fragment of LOX protein” or “LOX protein fragment” refers to a polypeptide having one or more (several) amino acids deleted from the amino and/or carboxyl terminus of the LOX protein; or a homologous sequence thereof; wherein the fragment has lysyl oxidase activity. Thus, variants of the LOX protein show lysyl oxidase activity. Assays for evaluating the lysyl oxidase activity of a given protein (or variant) are widely known in the state of the art. Examples of these assays include, without limited to, a method based on the measurement of tritiated water released by enzyme action from labeled protein-bound lysine and hydroxylysine (Melet, J. et al. 1977. Analytical Biochemistry, vol. 77(1): 141-146) and a fluorescent assay that utilizes 1,5-diaminopentane as substrate and released hydrogen peroxide which is detected using Amplex red in horseradish peroxidase-coupled reactions (Palamakumbura AH1 and Trackman PC. 2002. Anal Biochem, 300(2): 245-51). Additionally, commercial kits can also be used for measuring the lysyl oxidase activity, such as Lysyl Oxidase Activity Assay Kit from BioVision, Inc., San Francisco, or Abcam PLC, Cambridge, UK.

The ECM of the invention also comprises a bone morphogenetic protein-1 or BMP1. BMP1 is a protein which in humans is encoded by the BMP1 gene. The BMP1 locus encodes a protein that is capable of inducing formation of cartilage in vivo. BMP1 protein cleaves the C-terminal propeptides of procollagen I, II, and III and its activity is increased by the procollagen C-endopeptidase enhancer protein. The BMP1 gene is expressed as alternatively spliced variants that share an N-terminal protease domain but differ in their C-terminal region. There are five isoforms of the protein created by alternate splicing: isoform 3 precursor (NCBI Reference Sequence: NP_006120 (SEQ ID NO: 2)), isoform 1 precursor (NCBI Reference Sequence: NP_001190 SEQ ID NO: 3)), isoform X2 (NCBI Reference Sequence: XP_016869227, SEQ ID NO: 4)), isoform X3 (NCBI Reference Sequence: XP_011542919 (SEQ ID NO: 5)), isoform X1 (NCBI Reference Sequence: XP_006716449 (SEQ ID NO: 6)

```
SEQ ID NO: 2
    1 MPGVARLPLL LGLLLLPRPG RPLDLADYTY DLAEEDDSEP LNYKDPCKAA AFLGDIALDE
  61 EDLRAFQVQQ AVDLRRHTAR KSSIKAAVPG NTSTPSCQST NGQPQRGACG RWRGRSRSRR
121 AATSRPERVW PDGVIPFVIG GNFTGSQRAV FRQAMRHWEK HTCVTFLERT DEDSYIVFTY
181 RPCGCCSYVG RRGGGPQAIS IGKNCDKFGI VVHELGHVVG FWHEHTRPDR DRHVSIVREN
241 IQPGQEYNFL KMEPQEVESL GETYDFDSIM HYARNTFSRG IFLDTIVPKY EVNGVKPPIG
301 QRTRLSKGDI AQARKLYKCP ACGETLQDST GNFSSPEYPN GYSAHMHCVW RISVTPGEKI
361 ILNFTSLDLY RSRLCWYDYV EVRDGFWRKA PLRGRFCGSK LPEPIVSTDS RLWVEFRSSS
421 NWVGKGFFAV YEAICGGDVK KDYGHIQSPN YPDDYRPSKV CIWRIQVSEG FHVGLTFQSF
481 EIERHDSCAY DYLEVRDGHS ESSTLIGRYC GYEKPDDIKS TSSRLWLKFV SDGSINKAGF
541 AVNFFKEVDE CSRPNRGGCE QRCLNTLGSY KCSCDPGYEL APDKRRCEAA CGGFLTKLNG
601 SITSPGWPKE YPPNKNCIWQ LVAPTQYRIS LQFDFFETEG NDVCKYDFVE VRSGLTADSK
661 LHGKFCGSEK PEVITSQYNN MRVEFKSDNT VSKKGFKAHF FSDKDECSKD NGGCQQDCVN
721 TFGSYECQCR SGFVLHDNKH DCKEAGCDHK VTSTSGTITS PNWPDKYPSK KECTWAISST
781 PGHRVKLTFM EMDIESQPEC AYDHLEVFDG RDAKAPVLGR FCGSKKPEPV LATGSRMFLR
841 FYSDNSVQRK GFQASHATEC GGQVRADVKT KDLYSHAQFG DNNYPGGVDC EWVIVAEEGY
901 GVELVFQTFE VEEETDCGYD YMELFDGYDS TAPRLGRYCG SGPPEEVYSA GDSVLVKFHS
961 DDTITKKGFH LRYTSTKFQD TLHSRK
SEQ ID NO: 3
    1 MPGVARLPLL LGLLLLPRPG RPLDLADYTY DLAEEDDSEP LNYKDPCKAA AFLGDIALDE
  61 EDLRAFQVQQ AVDLRRHTAR KSSIKAAVPG NTSTPSCQST NGQPQRGACG RWRGRSRSRR
121 AATSRPERVW PDGVIPFVIG GNFTGSQRAV FRQAMRHWEK HTCVTFLERT DEDSYIVFTY
181 RPCGCCSYVG RRGGGPQAIS IGKNCDKFGI VVHELGHVVG FWHEHTRPDR DRHVSIVREN
241 IQPGQEYNFL KMEPQEVESL GETYDFDSIM HYARNTFSRG IFLDTIVPKY EVNGVKPPIG
301 QRTRLSKGDI AQARKLYKCP ACGETLQDST GNFSSPEYPN GYSAHMHCVW RISVTPGEKI
361 ILNFTSLDLY RSRLCWYDYV EVRDGFWRKA PLRGRFCGSK LPEPIVSTDS RLWVEFRSSS
421 NWVGKGFFAV YEAICGGDVK KDYGHIQSPN YPDDYRPSKV CIWRIQVSEG FHVGLTFQSF
481 EIERHDSCAY DYLEVRDGHS ESSTLIGRYC GYEKPDDIKS TSSRLWLKFV SDGSINKAGF
541 AVNFFKEVDE CSRPNRGGCE QRCLNTLGSY KCSCDPGYEL APDKRRCEAA CGGFLTKLNG
601 SITSPGWPKE YPPNKNCIWQ LVAPTQYRIS LQFDFFETEG NDVCKYDFVE VRSGLTADSK
661 LHGKFCGSEK PEVITSQYNN MRVEFKSDNT VSKKGFKAHF FSEKRPALQP PRGRPHQLKF
721 RVQKRNRTPQ
SEQ ID NO: 4
    1 MPGVARLPLL LGLLLLPRPG RPLDLADYTY DLAEEDDSEP LNYKDPCKAA AFLGDIALDE
  61 EDLRAFQVQQ AVDLRRHTAR KSSIKAAVPG NTSTPSCQST NGQPQRGACG RWRGRSRSRR
121 AATSRPERVW PDGVIPFVIG GNFTGSQRAV FRQAMRHWEK HTCVTFLERT DEDSYIVFTY
181 RPCGCCSYVG RRGGGPQAIS IGKNCDKFGI VVHELGHVVG FWHEHTRPDR DRHVSIVREN
241 IQPGQEYNFL KMEPQEVESL GETYDFDSIM HYARNTFSRG IFLDTIVPKY EVNGVKPPIG
301 QRTRLSKGDI AQARKLYKCP ACGETLQDST GNFSSPEYPN GYSAHMHCVW RISVTPGEKI
361 ILNFTSLDLY RSRLCWYDYV EVRDGFWRKA PLRGRFCGSK LPEPIVSTDS RLWVEFRSSS
421 NWVGKGFFAV YEAICGGDVK KDYGHIQSPN YPDDYRPSKV CIWRIQVSEG FHVGLTFQSF
481 EIERHDSCAY DYLEVRDGHS ESSTLIGRYC GYEKPDDIKS TSSRLWLKFV SDGSINKAGF
541 AVNFFKEVDE CSRPNRGGCE QRCLNTLGSY KCSCDPGYEL APDKRRCEAA CGGFLTKLNG
601 SITSPGWPKE YPPNKNCIWQ LVAPTQYRIS LQFDFFETEG NDVCKYDFVE VRSGLTADSK
661 LHGKFCGSEK PEVITSQYNN MRVEFKSDNT VSKKGFKAHF FSGGELFGLL GHPPRRP
SEQ ID NO: 5
    1 MPGVARLPLL LGLLLLPRPG RPLDLADYTY DLAEEDDSEP LNYKDPCKAA AFLGDIALDE
  61 EDLRAFQVQQ AVDLRRHTAR KSSIKAAVPG NTSTPSCQST NGQPQRGACG RWRGRSRSRR
121 AATSRPERVW PDGVIPFVIG GNFTGSQRAV FRQAMRHWEK HTCVTFLERT DEDSYIVFTY
181 RPCGCCSYVG RRGGGPQAIS IGKNCDKFGI VVHELGHVVG FWHEHTRPDR DRHVSIVREN
241 IQPGQEYNFL KMEPQEVESL GETYDFDSIM HYARNTFSRG IFLDTIVPKY EVNGVKPPIG
301 QRTRLSKGDI AQARKLYKCP ACGETLQDST GNFSSPEYPN GYSAHMHCVW RISVTPGEKI
361 ILNFTSLDLY RSRLCWYDYV EVRDGFWRKA PLRGRFCGSK LPEPIVSTDS RLWVEFRSSS
421 NWVGKGFFAV YEAICGGDVK KDYGHIQSPN YPDDYRPSKV CIWRIQVSEG FHVGLTFQSF
481 EIERHDSCAY DYLEVRDGHS ESSTLIGRYC GYEKPDDIKS TSSRLWLKFV SDGSINKAGF
541 AVNFFKEVDE CSRPNRGGCE QRCLNTLGSY KCSCDPGYEL APDKRRCEGC YDLQVGKPLL
601 WDRHCFRLST HGPEMLGTAL RG
SEQ ID NO: 6
    1 MPGVARLPLL LGLLLLPRPG RPLDLADYTY DLAEEDDSEP LNYKDPCKAA AFLGDIALDE
  61 EDLRAFQVQQ AVDLRRHTAR KSSIKAAVPG NTSTPSCQST NGQPQRGACG RWRGRSRSRR
121 AATSRPERVW PDGVIPFVIG GNFTGSQRAV FRQAMRHWEK HTCVTFLERT DEDSYIVFTY
181 RPCGCCSYVG RRGGGPQAIS IGKNCDKFGI VVHELGHVVG FWHEHTRPDR DRHVSIVREN
241 IQPGQEYNFL KMEPQEVESL GETYDFDSIM HYARNTFSRG IFLDTIVPKY EVNGVKPPIG
301 QRTRLSKGDI AQARKLYKCP ACGETLQDST GNFSSPEYPN GYSAHMHCVW RISVTPGEKI
361 ILNFTSLDLY RSRLCWYDYV EVRDGFWRKA PLRGRFCGSK LPEPIVSTDS RLWVEFRSSS
421 NWVGKGFFAV YEAICGGDVK KDYGHIQSPN YPDDYRPSKV CIWRIQVSEG FHVGLTFQSF
481 EIERHDSCAY DYLEVRDGHS ESSTLIGRYC GYEKPDDIKS TSSRLWLKFV SDGSINKAGF
541 AVNFFKEVDE CSRPNRGGCE QRCLNTLGSY KCSCDPGYEL APDKRRCEAA CGGFLTKLNG
601 SITSPGWPKE YPPNKNCIWQ LVAPTQYRIS LQFDFFETEG NDVCKYDFVE VRSGLTADSK
661 LHGKFCGSEK PEVITSQYNN MRVEFKSDNT VSKKGFKAHF FSGGLNGDQQ PLAQPLSADP
721 PGPGDLPFFV NHSALHPGSW RGQGTDTHIY QAWSYCSAPM PWSTLCPPHP QPCTETHTPT
781 RTHMCTHIAP SHKKPAEDPH WGHRGSALRA RGI
```

Any BMP1 protein isoform can be used in the context of the present invention, including additional members of the BMP1/tolloid-like (TLL) family, such as tolloid-like 1 (TLL1) or tolloid-like 2 (TLL2), which show a highly degree of homology with BMP1 in their catalytic N-terminal end. There are four isoforms of TLL1 protein created by alternate splicing, and one of TLL2: tolloid-like protein 1 isoform X1 (NCBI Reference Sequence: XP_016864059.1 (SEQ ID NO:7)), tolloid-like protein 1 isoform X2 (NCBI Reference Sequence: XP_011530516.1 (SEQ ID NO: 8)), tolloid-like protein 1 isoform 2 (NCBI Reference Sequence: NP_001191689.1 (SEQ ID NO: 9)), tolloid-like protein 1 isoform 1 (NCBI Reference Sequence: NP_036596.3 (SEQ ID NO: 10)), tolloid-like protein 2 precursor (NCBI Reference Sequence: NP_036597 (SEQ ID NO: 11)).

```
SEQ ID NO: 7
    1 MRGGQSQSVF WGDIALDDED LNIFQIDRTI DLTQNPFGNL GHTTGGLGDH AMSKKRGALY
  61 QLIDRIRRIG FGLEQNNTVK GKVPLQFSGQ NEKNRVPRAA TSRTERIWPG GVIPYVIGGN
121 FTGSQRAMFK QAMRHWEKHT CVTFIERSDE ESYIVFTYRP CGCCSYVGRR GNGPQAISIG
181 KNCDKFGIVV HELGHVIGFW HEHTRPDRDN HVTIIRENIQ PGQEYNFLKM EPGEVNSLGE
241 RYDFDSIMHY ARNTFSRGMF LDTILPSRDD NGIRPAIGQR TRLSKGDIAQ ARKLYRCPAC
301 GETLQESNGN LSSPGFPNGY PSYTHCIWRV SVTPGEKIVL NFTTMDLYKS SLCWYDYIEV
361 RDGYWRKSPL LGRFCGDKLP EVLTSTDSRM WIEFRSSSNW VGKGFAAVYE AICGGEIRKN
421 EGQIQSPNYP DDYRPMKECV WKITVSESYH VGLTFQSFEI ERHDNCAYDY LEVRDGTSEN
481 SPLIGRFCGY DKPEDIRSTS NTLWMKFVSD GTVNKAGFAA NFFKEEDECA KPDRGGCEQR
541 CLNTLGSYQC ACEPGYELGP DRRSCEAACG GLLTKLNGTI TTPGWPKEYP PNKNCVWQVV
601 APTQYRISVK FEFFELEGNE VCKYDYVEIW SGLSSESKLH GKFCGAEVPE VITSQFNNMR
661 IEFKSDNTVS KKGFKAHFFS DKDECSKDNG GCQHECVNTM GSYMCQCRNG FVLHDNKHDC
721 KEAECEQKIH SPSGLITSPN WPDKYPSRKE CTWEISATPG HRIKLAFSEF EIEQHQECAY
781 DHLEVFDGET EKSPILGRLC GNKIPDPLVA TGNKMFVRFV SDASVQRKGF QATHSTECGG
841 RLKAESKPRD LYSHAQFGDN NYPGQVDCEW LLVSERGSRL ELSFQTFEVE EEADCGYDYV
901 ELFDGLDSTA VGLGRFCGSG PPEEIYSIGD SVLIHFHTDD TINKKGFHIR YKSIRYPDTT
961 HTKK
SEQ ID NO: 8
    1 MFKQAMRHWE KHTCVTFIER SDEESYIVFT YRPCGCCSYV GRRGNGPQAI SIGKNCDKFG
  61 IVVHELGHVI GFWHEHTRPD RDNHVTIIRE NIQPGQEYNF LKMEPGEVNS LGERYDFDSI
121 MHYARNTFSR GMFLDTILPS RDDNGIRPAI GQRTRLSKGD IAQARKLYRC PACGETLQES
181 NGNLSSPGFP NGYPSYTHCI WRVSVTPGEK IVLNFTTMDL YKSSLCWYDY IEVRDGYWRK
241 SPLLGRFCGD KLPEVLTSTD SRMWIEFRSS SNWVGKGFAA VYEAICGGEI RKNEGQIQSP
301 NYPDDYRPMK ECVWKITVSE SYHVGLTFQS FEIERHDNCA YDYLEVRDGT SENSPLIGRF
361 CGYDKPEDIR STSNTLWMKF VSDGTVNKAG FAANFFKEED ECAKPDRGGC EQRCLNTLGS
421 YQCACEPGYE LGPDRRSCEA ACGGLLTKLN GTITTPGWPK EYPPNKNCVW QVVAPTQYRI
481 SVKFEFFELE GNEVCKYDYV EIWSGLSSES KLHGKFCGAE VPEVITSQFN NMRIEFKSDN
541 TVSKKGFKAH FFSDKDECSK DNGGCQHECV NTMGSYMCQC RNGFVLHDNK HDCKEAECEQ
601 KIHSPSGLIT SPNWPDKYPS RKECTWEISA TPGHRIKLAF SEFEIEQHQE CAYDHLEVFD
661 GETEKSPILG RLCGNKIPDP LVATGNKMFV RFVSDASVQR KGFQATHSTE CGGRLKAESK
721 PRDLYSHAQF GDNNYPGQVD CEWLLVSERG SRLELSFQTF EVEEEADCGY DYVELFDGLD
781 STAVGLGRFC GSGPPEEIYS IGDSVLIHFH TDDTINKKGF HIRYKSIRYP DTTHTKK
SEQ ID NO: 9
    1 MGLGTLSPRM LVWLVASGIV FYGELWVCAG LDYDYTFDGN EEDKTETIDY KDPCKAAVFW
  61 GDIALDDEDL NIFQIDRTID LTQNPFGNLG HTTGGLGDHA MSKKRGALYQ LIDRIRRIGF
121 GLEQNNTVKG KVPLQFSGQN EKNRVPRAAT SRTERIWPGG VIPYVIGGNF TGSQRAMFKQ
181 AMRHWEKHTC VTFIERSDEE SYIVFTYRPC GCCSYVGRRG NGPQAISIGK NCDKFGIVVH
241 ELGHVIGFWH EHTRPDRDNH VTIIRENIQP GQEYNFLKME PGEVNSLGER YDFDSIMHYA
301 RNTFSRGMFL DTILPSRDDN GIRPAIGQRT RLSKGDIAQA RKLYRCPACG ETLQESNGNL
361 SSPGFPNGYP SYTHCIWRVS VTPGEKVVFS LC
SEQ ID NO: 10
    1 MGLGTLSPRM LVWLVASGIV FYGELWVCAG LDYDYTFDGN EEDKTETIDY KDPCKAAVFW
  61 GDIALDDEDL NIFQIDRTID LTQNPFGNLG HTTGGLGDHA MSKKRGALYQ LIDRIRRIGF
121 GLEQNNTVKG KVPLQFSGQN EKNRVPRAAT SRTERIWPGG VIPYVIGGNF TGSQRAMFKQ
181 AMRHWEKHTC VTFIERSDEE SYIVFTYRPC GCCSYVGRRG NGPQAISIGK NCDKFGIVVH
241 ELGHVIGFWH EHTRPDRDNH VTIIRENIQP GQEYNFLKME PGEVNSLGER YDFDSIMHYA
301 RNTFSRGMFL DTILPSRDDN GIRPAIGQRT RLSKGDIAQA RKLYRCPACG ETLQESNGNL
361 SSPGFPNGYP SYTHCIWRVS VTPGEKIVLN FTTMDLYKSS LCWYDYIEVR DGYWRKSPLL
421 GRFCGDKLPE VLTSTDSRMW IEFRSSSNWV GKGFAAVYEA ICGGEIRKNE GQIQSPNYPD
481 DYRPMKECVW KITVSESYHV GLTFQSFEIE RHDNCAYDYL EVRDGTSENS PLIGRFCGYD
541 KPEDIRSTSN TLWMKFVSDG TVNKAGFAAN FFKEEDECAK PDRGGCEQRC LNTLGSYQCA
601 CEPGYELGPD RRSCEAACGG LLTKLNGTIT TPGWPKEYPP NKNCVWQVVA PTQYRISVKF
661 EFFELEGNEV CKYDYVEIWS GLSSESKLHG KFCGAEVPEV ITSQFNNMRI EFKSDNTVSK
721 KGFKAHFFSD KDECSKDNGG CQHECVNTMG SYMCQCRNGF VLHDNKHDCK EAECEQKIHS
781 PSGLITSPNW PDKYPSRKEC TWEISATPGH RIKLAFSEFE IEQHQECAYD HLEVFDGETE
841 KSPILGRLCG NKIPDPLVAT GNKMFVRFVS DASVQRKGFQ ATHSTECGGR LKAESKPRDL
901 YSHAQFGDNN YPGQVDCEWL LVSERGSRLE LSFQTFEVEE EADCGYDYVE LFDGLDSTAV
961 GLGRFCGSGP PEEIYSIGDS VLIHFHTDDT INKKGFHIRY KSIRYPDTTH TKK
SEQ ID NO: 11
    1 MPRATALGAL VSLLLLLPLP RGAGGLGERP DATADYSELD GEEGTEQQLE HYHDPCKAAV
  61 FWGDIALDED DLKLFHIDKA RDWTKQTVGA TGHSTGGLEE QASESSPDTT AMDTGTKEAG
121 KDGRENTTLL HSPGTLHAAA KTFSPRVRRA TTSRTERIWP GGVIPYVIGG NFTGSQRAIF
181 KQAMRHWEKH TCVTFIERTD EESFIVFSYR TCGCCSYVGR RGGGPQAISI GKNCDKFGIV
241 AHELGHVVGF WHEHTRPDRD QHVTIIRENI QPGQEYNFLK MEAGEVSSLG ETYDFDSIMH
301 YARNTFSRGV FLDTILPRQD DNGVRPTIGQ RVRLSQGDIA QARKLYKCPA CGETLQDTTG
361 NFSAPGFPNG YPSYSHCVWR ISVTPGEKIV LNFTSMDLFK SRLCWYDYVE VRDGYWRKAP
421 LLGRFCGDKI PEPLVSTDSR LWVEFRSSSN ILGKGFFAAY EATCGGDMNK DAGQIQSPNY
481 PDDYRPSKEC VWRITVSEGF HVGLTFQAFE IERHDSCAYD YLEVRDGPTE ESALIGHFCG
541 YEKPEDVKSS SNRLWMKFVS DGSINKAGFA ANFFKEVDEC SWPDHGGCEH RCVNTLGSYK
601 CACDPGYELA ADKKMCEVAC GGFITKLNGT ITSPGWPKEY PTNKNCVWQV VAPAQYRISL
661 QFEVFELEGN DVCKYDFVEV RSGLSPDAKL HGRFCGSETP EVITSQSNNM RVEFKSDNTV
721 SKRGFRAHFF SDKDECAKDN GGCQHECVNT FGSYLCRCRN GYWLHENGHD CKEAGCAHKI
781 SSVEGTLASP NWPDKYPSRR ECTWNISSTA GHRVKLTFNE FEIEQHQECA YDHLEMYDGP
841 DSLAPILGRF CGSKKPDPTV ASGSSMFLRF YSDASVQRKG FQAVHSTECG GRLKAEVQTK
901 ELYSHAQFGD NNYPSEARCD WVIVAEDGYG VELTFRTFEV EEEADCGYDY MEAYDGYDSS
961 APRLGRFCGS GPLEEIYSAG DSLMIRFRTD DTINKKGFHA RYTSTKFQDA LHMKK
```

In a particular embodiment of the ECM of the invention, the BMP1 protein comprises an amino acid sequence with an identity of at least 70, 75, 80, 85, 90, 95, 96, 97, 98, 99% with SEQ ID NO: 2.

The term “identity” has been defined in previous paragraphs. Thus, variants of the BMP1 protein of SEQ ID NO: 2 are encompassed within the context of the present invention. Apart from humans, other sources from which BMP1 protein variants can be obtained include, without limiting to, non-human animals such as non-human primates, pigs, mice and rats among others. In a more particular embodiment, the BMP1 protein comprises the sequence SEQ ID NO: 2. The present invention also encompasses fragments of the BMP1 protein, being said fragments considered variants of the BMP1 protein. As use herein, the term “fragment of BMP1 protein” or “BMP1 fragment” refers to a polypeptide having one or more (several) amino acids deleted from the amino and/or carboxyl terminus of the BMP1 protein; or a homologous sequence thereof; wherein the fragment has BMP1 activity. Consequently, the present invention also encompasses variants of the BMP1 protein showing the same function than BMP1 protein, i.e. cleaving the C-terminal propeptides of procollagen I, II, and III. Assays for evaluating the BMP1 activity of a given protein are widely known in the state of the art. Examples of assays for measuring the BMP1 activity include, without limited to, procollagen assay (Hartigan, N., Garrigue-Antar, L. and Kadler, K. E. 2003. The Journal of Biochemical Chemistry, 278(20): 18045-18049; pro-lysyl oxidase processing assay (Uzel M I, Scott I C, Babakhanlou-Chase H, Palamakumbura A H, Pappano W N, Hong H H, Greenspan D S, Trackman P C. J Biol Chem. 2001;276(25):22537-43). Additionally, commercial substrates can also be used for measuring BMP1 activity, such as the Recombinant Human BMP-1/PCP Assay from R&D Systems.

In a particular embodiment, the ECM of the invention comprises ECM producer cells. There are many cell types that contribute to the development of the various types of extracellular matrix. Fibroblasts are the most common cell type in connective tissue ECM, in which they synthesize, maintain, and provide a structural framework; fibroblasts secrete the precursor components of the ECM, including the ground substance. Chondrocytes are found in cartilage and produce the cartilagenous matrix. Osteoblasts are responsible for bone formation. Thus, in a particular embodiment of the ECM of the invention, the ECM producer cells are fibroblasts, keratinocytes, tenocytes, chondrocytes and/or any combination thereof. The cells are preferably harvested from a live human; though recently deceased human or animal donors may alternatively be used. In another particular embodiment, the ECM producer cells are fibroblasts. Fibroblasts may be obtained from a tendon of the patient. For example, a palmaris longus tendon could be removed from one arm of the patient. But for the addition of the ECM material, the harvested fibroblasts are isolated and cultured using standard techniques, for example, the harvested cells may be grown in Hamm's F-12 culture media, 10% fetal calf serum, L-glutamine (292.mu.g/cc), penicillin (100 u/cc), streptomycin (100.mu.g/cc), and ascorbic acid (5.mu.g/cc) at 37° C.

The ECM producer cells may be genetically modified in order to produce the LOX enzyme, or a fragment thereof, and the BMP1 protein, or a fragment thereof. Methods for genetically modified cells are widely known in the state of the art. Thus, in a particular embodiment of the ECM of the invention, the ECM producer cells are genetically modified for producing recombinant LOX enzyme and/or recombinant BMP1. This means that, in a particular embodiment, the ECM producer cells comprise overexpressed the nucleotide sequences encoding LOX enzyme and BMP1 protein, i.e. the amount of LOX enzyme and BMP1 produced by the cell is increased. The amino acid sequences encoding the LOX enzyme and BMP1 are disclosed below. As the skilled person knows, the overexpression of a gene may be achieved, for example, by increasing the number of copies of the genes encoding LOX enzyme and BMP1 protein, or by expressing these genes under the appropriate regulatory sequences such a promoter. The nucleotide sequences encoding the LOX enzyme and the BMP1 protein are known in the state of the art and can be retrieved from public databases.

In another particular embodiment, the ECM may further comprise other cells, for example stem cells, apart from those which commonly contribute to the development of the extracellular matrix. The ECM may be used as substrate for culturing cells, such us, without limiting to, mesenchymal stem cells, smooth muscle cells and cardiomyocytes. Thus, in a particular embodiment of the ECM of the invention, the ECM further comprises mesenchymal stem cells.

The ECM may also comprise a “bioactive agent” or a “bioactive compound”. These terms are used herein to refer to a compound or entity that alters, inhibits, activates or otherwise affects biological or chemical events. Example of bioactive agents may include, but are not limited to, osteogenic or chondrogenic proteins or peptides, anti-cancer substances, antibiotics, immunosuppressants, anti-viral substances, enzyme inhibitors, hormones, neurotoxins, opioids, hypnotics, anti-histamines, lubricants, anti-spasmodics and muscle contractants including channel blockers, miotics and anti-cholinergics, anti-parasite and/or anti-protozoal compounds, modulators of cell-extracellular matrix interactions including cell growth inhibitors and antiadhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, prostaglandins and anti-depressants. In certain embodiments, the bioactive agent is a drug. In certain embodiments, the bioactive agent is a small molecule. Bioactive agents further include RNAs, such as siRNA, and osteoclast stimulating factors. In some embodiments, the bioactive agent may be a factor that stops, removes, or reduces the activity of bone growth inhibitors. In some embodiments, the bioactive agent is a growth factor, cytokine, extracellular matrix molecule or a fragment or derivative thereof, for example, a cell attachment sequence such as RGD. In a particular embodiment, the bioactive agent is selected from transforming growth factor-beta (TGF-beta), dextran sulfate, ascorbate and combinations thereof. The ECM may comprise more than one bioactive agent. Thus, in another particular embodiment, the ECM further comprises a growth factor and extracellular matrix molecules. In a more particular embodiment, the ECM further comprises TGF-beta, dextran sulfate and ascorbate, being these bioactive agents capable of promoting the synthesis of extracellular matrix components. Other bioactive agents capable of promoting the synthesis of extracellular matrix components also include profibrotic cytokines such as tumor necrosis factor-alpha (TNF-alpha), bioactive peptides such as angiotensin II or proteins as connective tissue growth factor (CTGF). Molecules with capacity to increase the density of the extracellular medium such as Ficoll™ can also be comprised as bioactive agents.

Additionally, the ECM of the invention can be combined with autograft bone marrow aspirate, autograft bone, preparations of selected autograft cells, autograft cells containing genes encoding bone promoting action prior to being placed in a defect site.

In a particular embodiment, the extracellular matrix of the invention comprises an amount of insoluble collagen deposited into the matrix of, at least, 3 μg/10⁶ cells, more than four times the amount accumulated in the absence of LOX and BMP1 as can be seen in FIG. 4.

As explained at the beginning of the present description, the inventors have discovered that implementing fibroblast cultures with supernatants enriched in LOX and BMP1 from stable HEK293 cell lines strongly increased the deposition of collagen onto the insoluble matrix at the expense of the soluble fraction in the extracellular medium. Thus, in a particular embodiment, the ECM of the invention is decellularized.

Additionally, in another aspect, the present invention also encompasses a decellularized ECM comprising an increase amount of collagen in comparison with an ECM which has not been produced by culturing fibroblast with LOX and BMP1. Thus, the present invention relates to a decellularized extracellular matrix comprising an amount of insoluble collagen deposited into the matrix of, at least, 3 μg/10⁶ cells, more than four times the amount accumulated in the absence of LOX and BMP1.

### Uses of the Invention

Additionally, the authors of the present invention have also discovered that implementing fibroblast cultures with supernatants enriched in LOX and BMP1 strongly increased the deposition of collagen onto the insoluble matrix at the expense of the soluble fraction in the extracellular medium (Example 1). These results support a convenient protocol to enhance the capacity of in vitro cell cultures to deposit collagen in the extracellular matrix, which represents a promising approach for application in tissue engineering.

Thus, in another aspect, the present invention relates to an in vitro use of composition comprising a lysyl oxidase (LOX), or a fragment thereof, and bone morphogenetic protein-1 (BMP1), or a fragment thereof, hereinafter “first use of the invention”, for increasing the synthesis and/or deposit of collagen in an extracellular matrix.

The terms “Lysyl oxidase” and “bone morphogenetic protein-1” and fragment thereof have been defined in previous paragraphs. As explained above, in a particular embodiment, the LOX protein comprises an amino acid sequence with an identity of at least 70, 75, 80, 85, 90, 95, 96, 97, 98, 99% with SEQ ID NO: 1. In another particular embodiment, the BMP1 protein comprises an amino acid sequence with an identity of at least 70, 75, 80, 85, 90, 95, 96, 97, 98, 99% with SEQ ID NO: 4. The term “identity” has been defined above.

As can be seen in the examples of the present description, a composition comprising the LOX, or a fragment thereof, and BMP1, or a fragment thereof, hereinafter “composition of the invention”, can increase the synthesis and/or deposit of collagen in an extracellular matrix. This composition, further to comprise the LOX and BMP1 proteins, may comprise other type of compounds which favor the activity of LOX and BMP1. Examples of these compounds are any of the bioactive agents cited above. Thus, in a particular embodiment, the bioactive agent is selected from transforming growth factor-beta (TGF-beta), dextran sulfate, ascorbate and combinations thereof. The ECM may comprise more than one bioactive agent. Thus, in another particular embodiment, the ECM further comprises a growth factor and extracellular matrix molecules. In a more particular embodiment, the ECM further comprises TGF-beta, dextran sulfate and ascorbate.

In order to increase the synthesis and/or deposit of collagen in an extracellular matrix, the composition of the invention can be put into contact with the ECM producer cells and next these cells deposited on a surface to be cultured or, alternatively, the composition of the invention can be put directly into contact with a ECM or a surface as disclosed previously already comprising ECM producer cells.

Examples of ECM producer cells, which can be cultured together with the composition of the invention or which can be present in the ECM, include, but not limited to, fibroblast cells, keratinocyte cells, tenocyte cells, chondrocyte cells and/or any combination thereof. Thus, in a particular embodiment, the extracellular matrix comprises fibroblast cells, keratinocyte cells, tenocyte cells, chondrocyte cells and/or any combination thereof. The means and conditions (pH, medium, temperature, etc.) for culturing these cells are widely known in the state of the art.

As explained previously in the present invention, the inventors have discovered that fibroblast-derived matrices regulate adipogenic and osteogenic differentiation of human mesenchymal stem cells (MSC), and that this effect was modulated by LOX and BMP1 (see Example 2). These results provide evidence that fibroblast-derived matrices are able to regulate the adipogenic and osteogenic differentiation of human MSC, a powerful cell tool in regenerative medicine.

Thus, in another aspect, the present invention relates to an in vitro use of the extracellular matrix of the invention, hereinafter “second use of the invention”, for regulating the differentiation of stem cells, preferably mesenchymal stem cells, wherein the ECM is decellularized.

As used herein, the term “regulating” or “regulates” refers to control the capacity of the stem cells to differentiate into a more specialized cell type. In developmental biology, cellular differentiation is the process where a cell changes from one cell type to another. As the skilled person in the art knows, stem cells are a class of undifferentiated cells that are able to differentiate into specialized cell types. In a particular embodiment of the present invention, the stem cell whose differentiation is regulated by the ECM of the invention is a mesenchymal stem cell. Mesenchymal stromal/stem cells are a population of stromal cells present in the bone marrow, adipose and most connective tissues, capable of differentiation into mesenchymal tissues such as adipose tissue (adipogenesis), bone tissue (osteogenesis) and cartilage (chondrogenesis). The ECM of the invention may be used to culture MSCs and to regulate their differentiation into adipose, bone and cartilage cells. In a particular embodiment of the second use of the invention, the adipogenic and/or osteogenic differentiation of mesenchymal stem cells is regulated by the ECM of the invention, wherein the ECM is decellularized. In a more particular embodiment, the differentiation of mesenchymal stem cells is reduced or inhibited by the ECM of the invention, wherein the ECM is decellularized.

In the present description, it is considered that “the differentiation of mesenchymal stem cells is reduced” or “the differentiation of mesenchymal stem cells is inhibited” when more than 50%, preferably more than 60% of the whole cell population of mesenchymal stem cells do not differentiate into mesenchymal tissues.

As used herein, the term “decellularized” refers to the process used in biomedical engineering to isolate the ECM from its inhabiting cells, leaving an ECM scaffold. The skilled person in the art is able to kill the cells within the ECM without damaging the extracellular components. Physical, chemical and enzymatic methods can be used for removing the cells from an ECM. The most common physical methods used to lyse, kill, and remove cells from the matrix of a tissue through the use, for example, of temperature, force and pressure, and electrical disruption. Alternatively, to the physical methods, a proper combination of chemicals may be selected for decellularization depending on the thickness, extracellular matrix composition, and intended use of the ECM. The chemicals used to kill and remove the cells include, without limiting to, acids, alkaline treatments, ionic detergents, non-ionic detergents, and zwitterionic detergents. The ionic detergent, sodium dodecyl sulfate (SDS), is commonly used because of its high efficacy for lysing cells without significant damage to the ECM. Detergents act effectively to lyse the cell membrane and expose the contents to further degradation. After SDS lyses the cell membrane, endonucleases and exonucleases degrade the genetic contents, while other components of the cell are solubilized and washed out of the matrix. Alkaline and acid treatments can be effective companions with an SDS treatment due to their ability to degrade nucleic acids and solubilize cytoplasmic inclusions. The most well-known non-ionic detergent is Triton X-100, which is popular because of its ability to disrupt the interactions between lipids and between lipids and proteins. Triton X-100 does not disrupt protein-protein interactions, which is beneficial to keeping the ECM intact. EDTA is a chelating agent that binds calcium, which is a necessary component for proteins to interact with one another. By making calcium unavailable, EDTA prevents the integral proteins between cells from binding to one another. EDTA is often used with trypsin, an enzyme that acts as a protease to cleave the already existing bonds between integral proteins of neighboring cells within a tissue. Together, the EDTA-trypsin combination makes a good team for decellularizing ECM. Enzymes used in decellularization treatments are used to break the bonds and interactions between nucleic acids, interacting cells through neighboring proteins, and other cellular components. Lipases, thermolysin, galactosidase, nucleases, and trypsin have all been used in the removal of cells. After a cell is lysed with a detergent, acid, physical pressure, etc., endonucleases and exonucleases can begin the degradation of the genetic material.

Within the context of the present invention, it is also encompassed the use of the ECM of the invention for selecting medicaments in the treatment of diseases, as well as the use of the ECM of the invention as a support material for regenerating a biological tissue.

### Methods of the Invention

In another aspect, the invention relates to an in vitro method for obtaining an extracellular matrix of the invention, hereinafter “first method of the invention”, comprising incubating ECM producer cells in the presence of a composition comprising a lysyl oxidase (LOX), or a fragment thereof, and bone morphogenetic protein-1 (BMP1), or a fragment thereof, or culturing ECM producer cells genetically modified for producing the LOX enzyme, or a fragment thereof, and/or BMP1, or a fragment thereof.

The first method of the invention comprises incubating cells in the presence of a composition comprising LOX and BMP1 proteins or a fragment thereof. Methods and means for incubating cells are widely known in the state of the art. An example of these methods can be found in the illustrative examples of the present invention. Briefly, the ECM producer cells are incubated in DMEM medium (pH 7.4) without serum nor phenol red, and next dextran sulfate and ascorbate is added in presence or absence of TGF-beta for 4 days at 37° C.

Additionally, the composition further to comprise LOX and BMP1 proteins may also comprise a “bioactive agent” or a “bioactive compound”. These terms are used herein to refer to a compound or entity that alters, inhibits, activates or otherwise affects biological or chemical events. Examples of bioactive agent have been previously cited in the present description.

In a particular embodiment of the first method of the invention, the LOX protein comprises an amino acid sequence with an identity of at least 70, 75, 80, 85, 90, 95, 96, 97, 98, 99% with SEQ ID NO: 1. In a particular embodiment of the first method of the invention, the BMP1 protein comprises an amino acid sequence with an identity of at least 70, 75, 80, 85, 90, 95, 96, 97, 98, 99% with SEQ ID NO: 2. These particular embodiments, as well as the terms and expressions used, have been explained and defined in previous inventive aspects of the present invention.

In another particular embodiment, the ECM producer cells are fibroblasts, keratinocytes, tenocytes, chondrocytes and/or any combination thereof. In another particular embodiment, the ECM producer cells are genetically modified for producing the LOX enzyme, or a fragment thereof, and/or BMP1 or a fragment thereof.

After the cells have been incubated with the composition comprising LOX and BMP1 proteins, they may then be allowed to form an ECM in tissue culture or, after being loaded, onto a scaffold. In the first method of the invention for producing the ECM, the cells may be exposed to several factors or compounds to promote the production of said ECM. Once the ECM is produced, this may be isolated by techniques well known in the state of the art. The ECM thus obtained shows an increased deposit of collagen with respect to other ECM whose cells have not been incubated with a combination of LOX and BMP1 proteins.

Alternatively, the first method of the invention may comprise culturing ECM producer cells genetically modified for producing the LOX enzyme and/or BMP1. Examples of methods for genetically modifying cells have been disclosed previously in the present description.

As a consequence of putting into practice the first method of the invention, an ECM is obtained. Thus, in another aspect, the present invention relates to an ECM obtained by the first method of the invention.

In another aspect, the invention relates to an in vitro method for regulating the differentiation of stem cells, preferably mesenchymal stem cells, hereinafter “second method of the invention”, comprising culturing the stem cells, preferably mesenchymal stem cells (MSCs), in the extracellular matrix of the invention, wherein the ECM is decellularized . The term “decellularized” has been disclosed in previous paragraphs.

Substrates for conventional cell culture research include plastic, glass, and micro porous filters (e.g., cellulosic, nylon, glass fiber, polystyrene, polyester, and polycarbonate). Substrates for bio-reactors used in batch or continuous cell culture or in genetic engineering include hollow fiber tubes or micro carrier beads. In some embodiments, the substrate/container may be made of any suitable material capable of allowing the extracellular matrix components to adsorb or bind to at least one surface of the substrate or container. Such materials may include the following: cellulose, polystyrene, polycarbonate, polytetrafluoroethylene, nylon, glass, polyethyleneterephthalate, polymethylpentane, polypropylene, polyethylene and combinations thereof. Other materials that may be employed include Permanox®, polyester, polyamide, polyimide, and silica-based materials, including glass containers and the like. Combinations of any of the aforementioned materials may also be used. These materials may be porous or non-porous

The media used to culture the stem cells, preferably mesenchymal stem cells, is a conditioned or defined cell culture media. In one embodiment, the media is MEF-conditioned medium supplemented with basic fibroblast growth factor (bFGF). The bFGF may be present in an amount of about 4 to about 20 ng/ml in the media. It is noted, however, that the method of culturing is not limited to this culture medium. A high number of media have previously been shown to be compatible with culturing MSC cells.

By way of illustration, conditioned medium can be prepared by culturing irradiated or mitomycin C-inactivated primary mouse embryonic fibroblasts in a serum replacement medium such as, for example, DMEM, K/O DMEM, or DMEM/T12 containing 4 ng/mL basic fibroblast growth factor (bFGF). The culture supernatant is typically harvested after 1 day at 37° C., and supplemented with additional growth factors, including BFGF. More specifically, feeder-free culture, suitable base media can be made from the following components: Dulbecco's modified Eagle's medium (DMEM), Knockout Dulbecco's modified Eagle's medium (KG DMEM), Ham's F12/50% DMEM basal medium; 200 mM L-glutamine, non-essential amino acid solution, 3-mercaptoethanol, human recombinant basic fibroblast growth factor (bFGF).

The media/medium is then combined with the cells used for conditioning in an environment that allows the cells to release into the medium the components that support stem cells. Optionally, the cells can be inactivated (i.e., rendered incapable of substantial replication) by radiation (e.g., about 4,000 rads), treatment with a chemical inactivator like mitomycin c, or by any other effective method. The inactivation of the cells is not necessary in instances where the medium is separated from the conditioning cells before use in supporting stern cell cultures. The cells are cultured in the medium for sufficient time to allow adequate concentration of released factors (or consumption of media components) to produce a medium that supports the culturing of embryonic stem cells without differentiation. Typically, medium conditioned by culturing for 24 h at 37° C. produces medium that supports stem cell culture for 24 hours. However, the culturing period can be adjusted upwards or downwards, determining empirically (or by assaying for the concentration of essential factors) what constitutes an adequate period.

The stem cells, preferably mesenchymal stem cells, can be plated onto the ECM of the invention in a suitable distribution and in the presence of the conditioned medium.

A convenient way to determine whether MSCs are differentiating is to follow the morphological features of the colonies. For example, characteristic morphological features of undifferentiated MSCs are known by those skilled in the art, and include high nuclear/cytoplasmic ratios, prominent nucleoli, and compact colony formation with poorly discernable cell junctions. During passage, some cells may differentiate (particularly when replated as single cells, or when large clusters are allowed to form). However, cultures typically reestablish a larger proportion of undifferentiated cells during the culture period. Ideally, the propagated cells will have a doubling time of no more than about 20-40 hours.

The present invention also relates to a method of maintaining and expanding stem cells, preferably mesenchymal stem cells, in culture in an undifferentiated state, the method comprising culturing the mesenchymal stem cells in the extracellular matrix of the invention wherein the ECM is decellularized.

In another aspect, the present invention relates to a method for increasing the deposit of collagen in an extracellular matrix, hereinafter “third method of the invention”, comprising cultivating cells in the presence of a composition comprising a lysyl oxidase (LOX) and bone morphogenetic protein-1 (BMP1), or culturing ECM producer cells genetically modified for producing the LOX enzyme and/or BMP1.

In a particular embodiment of the third method of the invention, the LOX protein comprises an amino acid sequence with an identity of at least 70, 75, 80, 85, 90, 95, 96, 97, 98, 99% with SEQ ID NO: 1.

In a particular embodiment of the third method of the invention, the BMP1 protein comprises an amino acid sequence with an identity of at least 70, 75, 80, 85, 90, 95, 96, 97, 98, 99% with SEQ ID NO: 2.

In a particular embodiment, the ECM producer cells are fibroblasts, keratinocytes, tenocytes, chondrocytes and/or any combination thereof.

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples and drawings are provided by way of illustration and are not intended to be limiting of the present invention.

### BRIEF DESCRIPTION OF THE DRAWINGS

FIG. 1. Time-dependent stimulation of collagen synthesis and deposition in fibroblasts incubated with and without TGF-β1. A) Soluble collagen in the supernatant. B) Pepsin-solubilized collagen fraction associated to cell monolayer. C) Insoluble collagen deposited into the matrix. Collagen fractions were determined from cells incubated for 1 to 4 days in the absence (white bars) or presence of 5 ng/ml TGF-Iβ1 (black bars) as described under Materials and Methods. Values are represented as μg collagen per million of cells (mean±SEM, n=6; *P<0.05 or **P<0.01 vs one day in the absence of TGF-Iβ1, and #P<0.05 vs the corresponding time value in the absence of TGF-β1).

FIG. 2. Generation of HEK293 cells overexpressing secreted and active forms of LOX and BMP1 proteins. Induction of LOX (A) and BMP1 (B) proteins in HEK293 cells upon incubation with the tetracycline analog, doxycycline, at 10 μM as assessed by western blotting using total extracts (left panel) or Amicon-concentrated aliquots of the cell supernatant (right panel). C) Combination of cell supernatants containing LOX and BMP1 proteins gives rise to the proteolytic activation of LOX as assessed by western blotting. The blots shown correspond to representative experiments performed twice with two independent preparations. LOX-immunoreactive bands from results shown in panel C were quantified and expressed as percentage of total: 50 KDa precursor (open circle), 30 KDa active form (closed circle), and 25 KDa unknown band (open squares). D) LOX enzymatic activity as measured using Amplex red assay in cell supernatants from uninduced cells (Basal, white bar), induced and without BMP1 (Only LOX, closed bar), or induced and combined with BMP1 supernatants for 5-60 minutes (LOX+BMP1, closed bars). Values are represented as arbitrary fluorescent units (mean±SEM, n=6; *P<0.05, **P<0.01).

FIG. 3. LOX immunoreactivity in the supernatants of fibroblast cultures supplemented with LOX- and BMP1-containing conditioned media. LOX, BMP1 or both LOX/BMP1 supernatants were added to fibroblast cultures in the presence (T) or absence (basal, B) of TGF-β1 and LOX immunoreactivity assessed by western blotting at the beginning of the experiment (A, one day) or at the end (B, four days). The blots shown correspond to representative experiments performed twice with two independent preparations.

FIG. 4. Effect of the supplementation with LOX/BMP1 supernatants on collagen deposition from fibroblast cultures. Collagen fractions as measured in FIG. 2 were analyzed in fibroblasts exposed to conditioned media from control or LOX- and BMP1-overexpressing cells and incubated with and without TGF-β1 for 4 days. A) Soluble collagen in the supernatant. B) Pepsin-solubilized collagen fraction associated to cell monolayer. C) Insoluble collagen deposited into the matrix. D) LOX-derived pyridinoline (PYD) cross-link levels in the deposited matrix from fibroblast cultures exposed to conditioned media as assessed by specific ELISA. Values are represented as μg collagen or concentration of PYD per million of cells (mean±SEM, n=6; *P<0.05 vs the corresponding control values with TGF-β1).

FIG. 5. Collagen type I immunoreactivity in the supernatants of fibroblast cultures supplemented with LOX- and BMP1-containing conditioned media. Fibroblast cultures were exposed to control or LOX/BMP1 supernatants in the presence (T) or absence (B) of TGF-β1 for 4 days and collagen type I immunoreactivity assessed by western blotting as described under Materials and Methods. Specific collagen type I immunoreactivity was detected as a TGF-β1-induced band of approximately 150 KDa. The blots shown correspond to representative experiments performed twice with two independent preparations.

FIG. 6. Immunofluorescence analysis of collagen type I deposition from fibroblast cultures exposed to LOX/BMP1 supernatants. Fibroblasts exposed to control or LOX/BMP1 supernatants and incubated in the presence of TGF-β1 for 4 days were processed for immunofluorescence analysis of collagen type I as described under Materials and Methods. Micrographs shown correspond to representative results of staining for collagen type I (left column) and nuclei using DAPI (right column) performed twice with two independent preparations.

FIG. 7. Immunofluorescence detection of deposited collagen I in decellularized matrices from fibroblasts exposed to LOX/BMP1 supernatants. Fibroblast monolayers exposed to control or LOX/BMP1 supernatants in the presence of TGF-β1 for 4 days were decellularized before processing for immunofluorescence analysis of collagen type I as described under Materials and Methods. Micrographs shown correspond to representative results of staining for collagen type I (left column) performed twice with two independent preparations. The absence of DAPI staining confirmed the effectiveness of the decellularization procedure.

FIG. 8. Adipogenic differentiation of human MSC seeded on decellularized matrices from fibroblasts exposed to LOX/BMP1 supernatants. Adipogenic capacity was evaluated by microscopic examination (A) and quantified by spectrophotometric analysis (B) using Oil Red O staining in human MSC seeded without matrix, with matrix from TGF-β-stimulated fibroblasts exposed to control medium or with LOX/BMP1. Micrographs shown correspond to representative results of staining performed twice with two independent preparations. Values are represented as absorbance at 540 nm (mean±SEM, n=6; *P<0.05 vs no matrix, and #P<0.05 vs matrix fibroblast-derived matrix under control medium).

FIG. 9. Osteogenic differentiation of human MSC seeded on decellularized matrices from fibroblasts exposed to LOX/BMP1 supernatants. The capacity of human MSC to differentiate into osteoblasts in substrates without matrix, with matrix from TGF-β1 stimulated fibroblasts exposed to control medium or with LOX/BMP1was assessed by microscopic examination (A) and quantified by spectrophotometry (B) using Alizarin Red S staining. Micrographs shown correspond to representative results of staining performed twice with two independent preparations. Values are represented as absorbance at 405 nm (mean±SEM, n=6; *P<0.05 vs no matrix, and #P<0.05 vs matrix fibroblast-derived matrix under control medium).

### EXAMPLES

### I. Material and Methods

### Fibroblast Cell Culture

The human fibroblast cell line CCD-19Lu (ATCC) was maintained in culture medium as already described (Puig et al., 2015, Molecular Cancer Research 13, 161-173). For collagen analysis, fibroblasts were seeded on 100-mm dishes in culture medium without serum and phenol red but containing 100 μg/ml 500 KDa dextran sulfate (DxS) and 29 μg/ml L-ascorbic acid 2-phosphate (Sigma-Aldrich, St. Louis, Mo.), for up to four days in the absence or presence of 5 ng/ml TGF-β1 (R&D Systems, Minneapolis, Minn.).

### Collagen Analysis

At the end of the experimental time, culture media were collected and soluble collagen measured upon concentration with Sircol Soluble Collagen Assay (Biocolor, Carrickfergus, United Kingdom) following manufacturer's instructions. Cell layers were scrapped, extracted overnight with acid-based buffer (0.5 M acetic acid), and resulting pellets digested with 0.5 mg/ml pepsin (Sigma-Aldrich) in 10 mM HCl. Corresponding solubilized fractions were analyzed for collagen with Sircol. Insoluble collagen after pepsin digestion was hydrolyzed at 100° C. for 16 hours with 12 M HCl, neutralized with NaOH and analyzed by hydroxyproline assay using hydrolyzed type I collagen as standard (Kesava Reddy and Enwemeka, 1996, Clinical Biochemistry 29, 225-229.). Hydrolyzed fractions were also assayed for the content of the pyridinoline cross-links (PYD) using a commercially available ELISA kit (Quidel, Athens, Ohio).

Soluble collagen in the supernatant was also analyzed by western blotting using an specific anti-collagen al type I antibody (sc-8784, Santa Cruz, Dallas, Tex.) upon protein fractionation in sodium dodecyl sulphate-polyacrylamide gel electrophoresis (SDS-PAGE) following protocols previously described (Busnadiego et al., 2013, Molecular and Cellular Biology 33, 2388-2401).

### Generation of HEK293 Cell Clones Overexpressing LOX and BMP1

A full-length human LOX construct in pYX-Asc vector was obtained from Imagenes GmbH (Berlin, Germany). A full-length human BMP1 construct in pBabe vector was kindly provided by Victor L. Ruiz-Pérez (Instituto de Investigaciones Biomédicas “Alberto Sols”, Madrid, Spain) (Martínez-Glez et al., 2012, Human Mutation 33, 343-350). Both constructs were cloned into the vector pcDNA5/FRT/TO (Invitrogen, Carlsbad, Calif.), to obtain the corresponding pcDNA5/FRT/TO-LOX and -BMP1 plasmids. These constructs were then co-transfected with the Flp recombinase expression plasmid pOG44 into the Flp-In T-REx 293 cell line using Lipofectamine 2000 (Invitrogen). These cells stably express the Tet repressor and contain a single integrated FRT (Flp recombination target) site. Flp recombinase expression from the pOG44 vector mediates insertion of the cDNA cassettes into the genome at the integrated FRT site through site-specific DNA recombination. After 48 hours, cells were selected for hygromycin B resistance (Roche Diagnostics, Barcelona, Spain), and clones appeared after 10-15 days. Isogenic pooled clones were expanded and checked for transgene expression after 48 hours of incubation in the absence or presence of the tetracycline analog, doxycycline at 10 μM. Culture media were concentrated using Amicon Ultra-4 centrifugal filter units (Ultra-Cel 10K, Millipore, Cork, Ireland). LOX and BMP1 protein levels in cell layers or concentrated supernatants were detected by western blotting using specific antibodies against LOX (ab31238, Abcam, Cambridge, United Kingdom) and BMP1 (AF1927, R&D Systems). LOX enzymatic activity was determined using a commercially available assay from Abcam.

### Immunofluorescence Studies

Fluorescence microscopy was performed as previously described (Lagares et al., 2012). Briefly, cells were seeded onto 10 mm glass diameter coverslips (No. 1.5) in 35 mm culture dishes (Mattek, Ashland, Mass.). After the corresponding treatment, cells were fixed with cold methanol for 5 minutes, blocked with 1% BSA in phosphate-buffered solution (PBS) for 1 h, and then incubated overnight at 4° C. with anti-collagen al type I antibody (Santa Cruz), followed by the corresponding fluorescent secondary antibodies. Cell fluorescence was visualized by microscopy with a Nikon Eclipse T2000U (Nikon, Amstelveen, The Netherlands).

For analysis of the matrix deposited from cells, decellularization was performed by incubation with an extraction buffer containing 0.5% (v/v) Triton X-100 and 20 mM NH₄OH in PBS for 3-5 minutes as previously described (Cukierman, 2001, Preparation of Extracellular Matrices Produced by Cultured Fibroblasts, Current Protocols in Cell Biology. John Wiley & Sons, Inc.).

### Analysis of Adipogenic and Osteogenic Differentiation of Human Mesenchymal Stem Cells

Human mesenchymal stem cells (MSC) (Promocell, Heidelberg, Germany) were maintained in culture under basal medium (Promocell) and then induced for adipogenesis and osteogenesis with corresponding differentiation media (Promocell) for 21 days with medium change every 2-3 days. Phenotypic changes induced by lineage differentiation, i.e. the formation of lipid vesicles for adipogenesis and the extracellular deposition of calcium phosphate for osteogenesis, were monitored by staining with Oil Red O and Alizarin Red S (Santa Cruz), respectively, as described previously (Bruedigam et al., 2007, Basic Techniques in Human Mesenchymal Stem Cell Cultures: Differentiation into Osteogenic and Adipogenic Lineages, Genetic Perturbations, and Phenotypic Analyses, Current Protocols in Stem Cell Biology. John Wiley & Sons, Inc.). Differentiation was assessed by microscopic examination and quantitatively determined by spectrophotometric analysis upon dye solubilization.

### Statistical Analysis

Experimental data were analyzed using the unpaired Student t test in the case of normal distribution of data or using nonparametric tests as appropriate. The P values obtained are indicated in the figure legends when statistically significant (P<0.05).

### II. Results

### Example 1: Composition Comprising Lysyl Oxidase (LOX) and Bone Morphogenetic Protein-1 (BMP1) Strongly Increases Collagen Deposition In Vitro

### In Vitro Collagen Deposition is Slow and has Low Efficiency

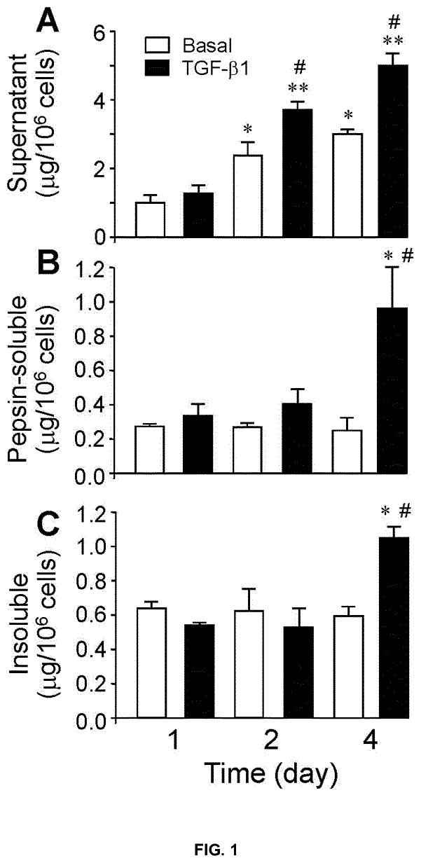

We have studied the synthesis and deposition of collagen in cultures of human lung fibroblast (CCD19-Lu) cells under basal conditions or incubated with the profibrotic cytokine transforming growth factor (TGF)-β1 for time periods ranging from one to four days (FIG. 1). Several fractions of collagen can be extracted from the cultures representing the sequential steps in the biosynthetic process. Cell supernatants were assayed for the soluble form of secreted collagen. Acid-based buffer was used to extract recently deposited, non-crosslinked collagen in cell monolayers. Pepsin treatment was then used to proteolytically digest the non-collagenous telopeptide segments, and thereby, solubilize recently cross-linked collagen. A Sirius-based colorimetric assay was used to determine collagen made soluble by these procedures. Insoluble collagen in the cell pellets was finally hydrolyzed with strong acid and heat and hydroxyproline measured as an estimation of heavily cross-linked collagen. As shown in FIG. 1A, soluble collagen progressively accumulated in cell supernatants from fibroblasts incubated under basal conditions, and this effect was further augmented in cells stimulated with TGF-131. In spite of this rate of synthesis and accumulation of soluble collagen, deposition into the matrix as pepsin-soluble or insoluble forms only modestly increased in cells incubated for four days with TGF-β1 (FIG. 1B and FIG. 1C). Acid-based buffer solubilized negligible amounts of collagen, indicating this pool is not stable in our experimental conditions (data not shown). Overall, these results indicate that, despite an active production and secretion of collagen precursors, in vitro deposition is an unfavoured process, even in conditions of macromolecular “crowding”, such as those used in our study.

### Generation of HEK293 Cell Lines Overexpressing Lysyl Oxidase (LOX) and Bone Morphogenetic Protein-1 (BMP1)

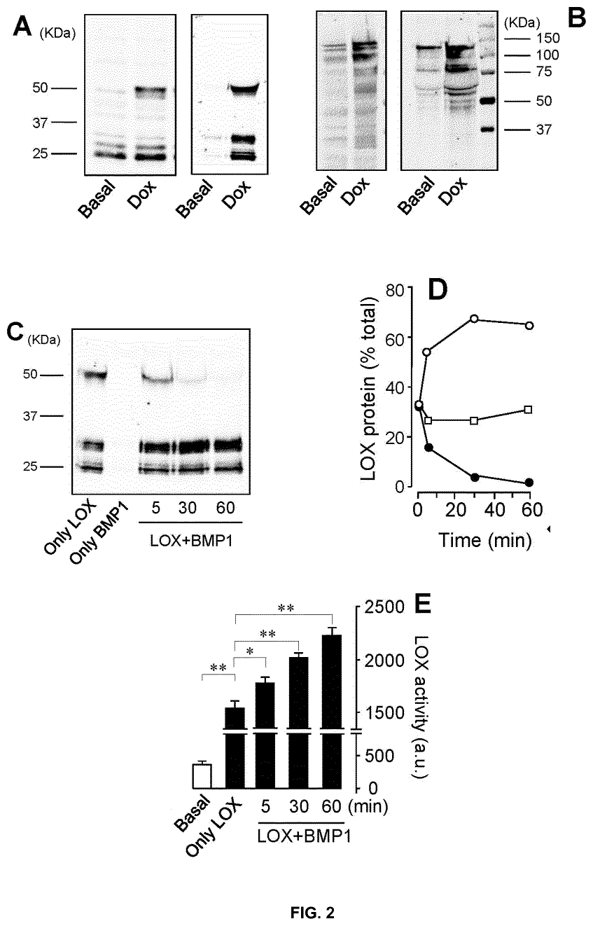

Several evidences in the literature suggest that an incomplete conversion of procollagen by C-proteinase/bone morphogenetic protein 1 (BMP1) significantly limits collagen deposition in vitro. Among several matrix (and non-matrix) substrates, BMP1 also catalyzes the proteolytic conversion of the precursor of lysyl oxidase (LOX) to yield the active form, thereby promoting the initial step in the process of collagen cross-linking. We hypothesized that the addition of LOX and/or BMP1 may represent a strategy to boost in vitro deposition of collagen. For that purpose, we generated HEK293 cell clones stably expressing LOX and BMP1 constructs under tetracyclin-dependent control. As shown in FIG. 2A, LOX transfectants expressed and secreted to the extracellular medium several LOX immunoreactive bands including the precursor of about 50 KDa, and shorter bands of 25 and 30 KDa. In a similar approach, BMP1 transfectants showed doxycycline-sensitive expression and secretion of a complex mixture of BMP1 forms ranging from 60-100 KDa, likely representing precursor and processed forms (FIG. 2B). The presence in LOX-overexpressing cell of the 50 KDa band of LOX indicates a limited capacity of the cells to process and activate the enzyme. Interestingly, incubation of cell supernatants containing LOX with those with BMP1 promoted the proteolysis of the precursor pro-LOX to the active form of 30 KDa in a time-dependent manner (FIG. 2C and D). The shortest LOX form of 25 KDa was not modified by the action of BMP1. LOX enzymatic activity was assessed in a fluorometric assay using supernatants from basal and doxycycline-incubated cells. As shown in FIG. 2E, the induction of the expression of LOX promoted a strong increase in LOX enzymatic activity, that was further augmented upon incubation with BMP1 supernatants in a time-dependent manner. Taken together, we succeed in generating HEK293-based cell systems to produce supernatants enriched with LOX and BMP1 enzymes which, when combined together, recapitulated in vitro the proteolytic activation of LOX.

### Addition of Recombinant Lysyl Oxidase (LOX) and Bone Morphogenetic Protein-1 (BMP1) Strongly Increases Collagen Deposition In Vitro

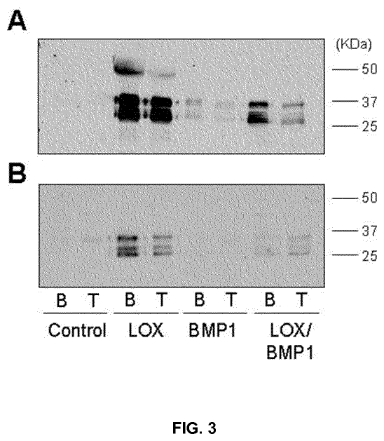

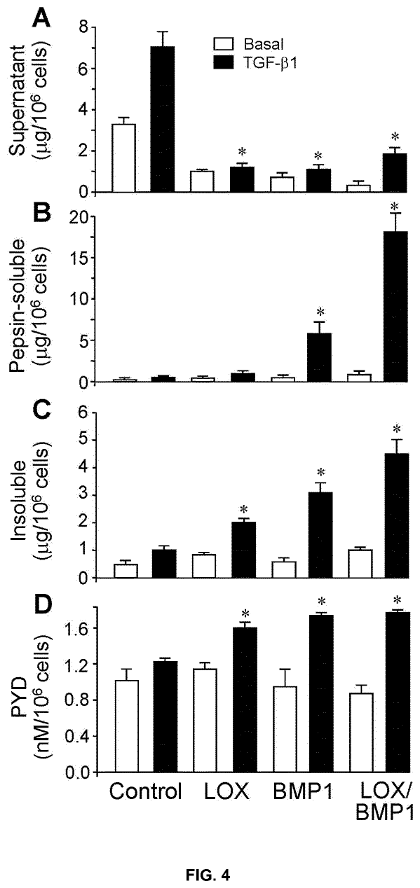

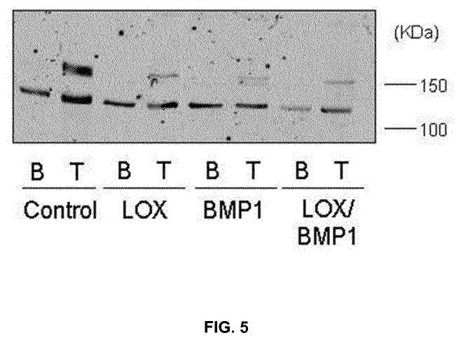

We have first checked the proteolytic activation of LOX in fibroblasts exposed to supernatants. As shown in FIG. 3A, fibroblasts incubated for one day with only LOX supernatants displayed a significant amount of the unprocessed LOX precursor, again indicating a limited cell capacity to in vitro process the proenzyme. In contrast, the combination of recombinant LOX and BMP1 resulted in complete proteolysis of the pro-LOX. The presence of processed forms of LOX in fibroblasts incubated with BMP1 alone indicated that the protease promoted the processing of endogenously produced LOX. No detectable LOX bands were observed in fibroblasts exposed to control media. After four days of incubation with supernatants, proteolytic conversion of pro-LOX enzyme was complete, even in the absence of added BMP1 (FIG. 3B). Interestingly, LOX immunoreactive signals were lower in supernatants from LOX/BMP1 than those from only LOX (at both one and four days), as well as in LOX (or LOX/BMP1) at one day compared to corresponding samples at four days, indicating that as soon as the processed forms of LOX are generated, they are either degraded or retained into the matrix. We have then studied the effect of these supernatants on collagen synthesis and deposition. As shown in FIG. 4A, as opposed to cells exposed to control media, the incubation of fibroblasts with cell supernatants containing either LOX, BMP1 or a mixture of both abrogated the accumulation of soluble collagen in the extracellular medium, both in the absence or presence of TGF-β1, an effect that was further corroborated by immunoblotting using an anti-col1α1 antibody (FIG. 5). Concomitantly with this drastic reduction, both pepsin-soluble and -insoluble fractions from TGF-β1-treated cells were found to significantly increase, being higher in cells incubated with the mixture of LOX/BMP1 than with either only LOX or BMP1, an observation that suggests a synergic action for the effect of both enzymes (FIG. 4B and C). LOX enzyme catalyzes the oxidative deamination of telopeptide lysine/hydrolysine residues to yield highly reactive aldehydes that further react to form immature and then mature permanent cross-links. The preferential use of hydroxylysine versus lysine in cross-linking reactions determines a distinctive pattern of maturational products, with higher levels of pyridinolines than of pyrroles, as is usually found in cartilage, bone or aorta. Hydrolyzed pepsin-insoluble pellets were assayed with a specific ELISA for the presence of pyridinoline cross-links (PYD). As shown in FIG. 4D, compared with control, the exposure of fibroblasts to LOX and/or BMP1 supernatants promoted the formation of PYD cross-links, indicating that a significant part of the deposited collagen is formed through this maturation pathway.

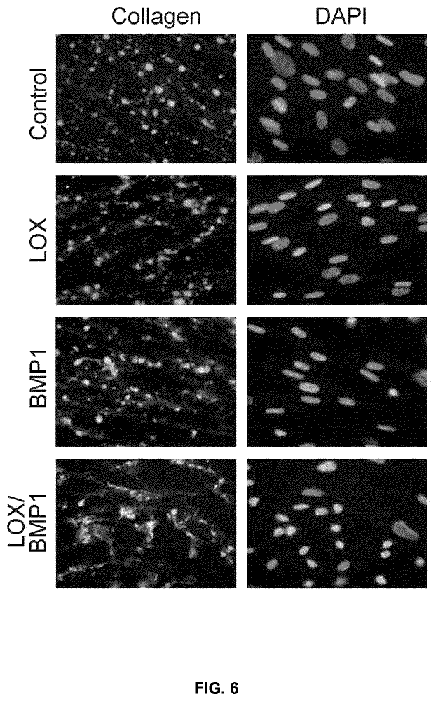

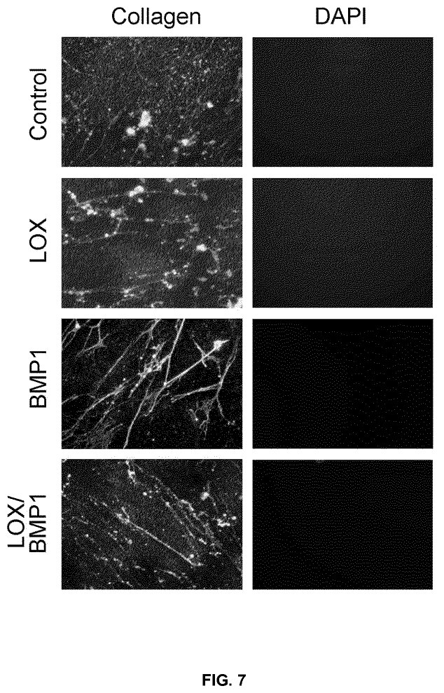

We have also analyzed the effect of LOX and BMP1-containing supernatants by immunofluorescence analysis using an anti-collal antibody. As shown in FIG. 6, fibroblasts exposed to control media displayed collagen type I immunoreactivity in the form of small and large aggregates. While this appearance was not significantly modified by LOX supernatants, cells exposed to BMP1 and particularly to the mixture of BMP1 and LOX showed a more distinctive pattern of immunoreactivity that includes the presence of fibrous material, likely consistent with their deposition to the matrix, rather than associated with the cell layer. This was further corroborated with experiments in decellularized matrices. As shown in FIG. 7, upon removal of the cell-associated material, a more fibrous pattern was observed in deposited matrix from cells exposed to BMP1 and the mixture of BMP1 and LOX. DAPI staining confirmed that the extraction procedure efficiently removed the cell layer. Taken together, our results show that the implementation of fibroblast cultures with supernatants enriched in LOX and BMP1 was an effective approach to strongly increase the deposition of collagen onto the insoluble matrix.

### Example 2: Fibroblast-Derived Matrix Modified by Lysyl Oxidase (LOX) and Bone Morphogenetic Protein-1 (BMP1) Regulates the Differentiation of Human Mesenchymal Stem Cells (MSC)

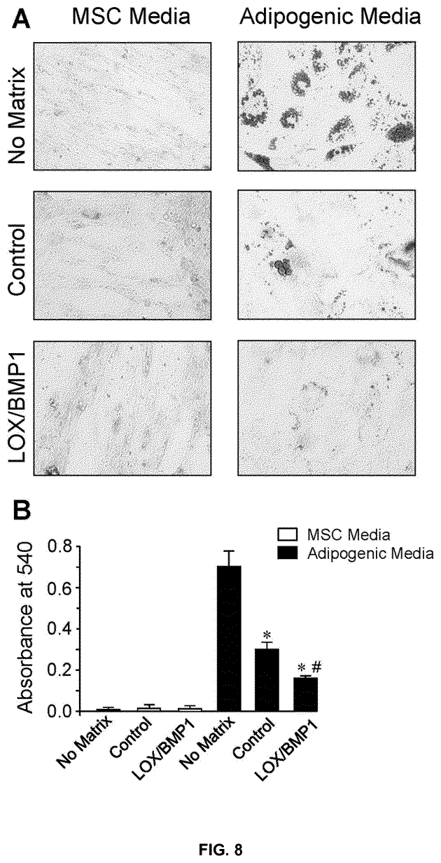

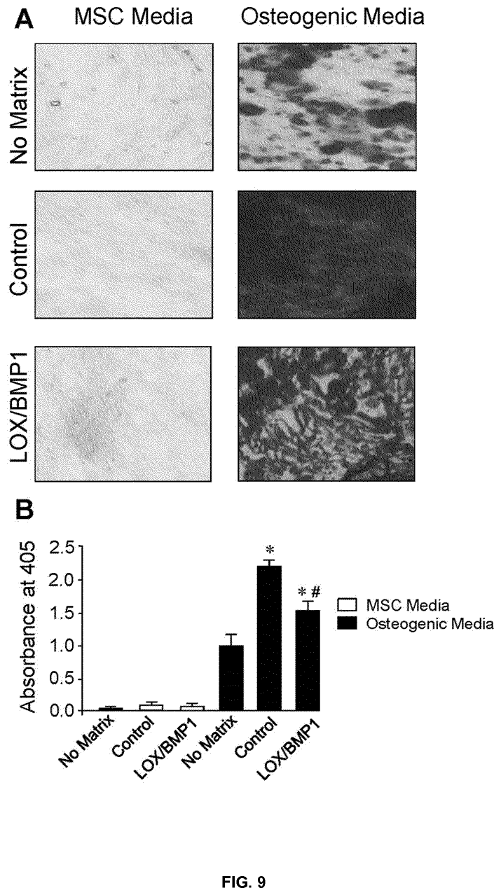

Mesenchymal stem cells are a promising source for regenerative medicine due to its capacity to self-renew and to differentiate into various tissue lineages, such as adipocytes, osteoblasts, and chondrocytes. Since the ECM provides physical and chemical cues to regulate MSC activities, we investigated the effects of fibroblast-derived matrix modified by LOX/BMP1 on regulating MSC differentiation to adipogenic and osteogenic lineages. For that purpose, we exposed fibroblast cultures to control media or to LOX and BMP1-containing supernatants as described above, then cells were removed and deposited matrix used as a substrate to establish MSC cultures. Once these cultures reached confluence, they were induced into adipogenic and osteogenic lineages by incubation with the corresponding differentiation media. These cultures were then compared with equivalent MSC seeded without any matrix. As shown in FIG. 8, after 14 days under adipogenic differentiation medium MSC without matrix develop lipid droplets that can be visualized with Oil Red O. MSC cultured on matrices derived from fibroblasts exposed to control media showed a reduced capacity to differentiate to adipocytes, and this behavior was further exacerbated in matrices from fibroblasts incubated with LOX/BMP1. On the other hand, MSC differentiation into osteogenic lineage results in the formation of extracellular calcium deposits that can be specifically stained using Alizarin Red S, as shown in FIG. 9 for MSC without matrix. Osteogenic differentiation was strongly enhanced in MSC seeded on matrices from fibroblasts exposed to control media, this effect being attenuated in matrices from fibroblasts incubated with LOX/BMP1 supernatants. These results indicate that fibroblast-derived matrix is able to regulate adipogenic and osteogenic differentiation capacity of MSC, being the modification promoted by LOX/BMP1 capable to fine-tune this ability.

1. An extracellular matrix (ECM) comprising a lysyl oxidase (LOX), or a fragment thereof, and bone morphogenetic protein-1 (BMP 1), or a fragment thereof.

2. An extracellular matrix according to claim 1, wherein LOX protein comprises an amino acid sequence with an identity of at least 70, 75, 80, 85, 90, 95, 96, 97, 98, 99% with SEQ ID NO: 1.

3. An extracellular matrix according to claim 1, wherein BMP1 protein comprises an amino acid sequence with an identity of at least 70, 75, 80, 85, 90, 95, 96, 97, 98, 99% with SEQ ID NO: 2.

4. An extracellular matrix according to claim 1, wherein the ECM further comprises ECM producer cells.

5. An extracellular matrix according to claim 1, wherein the ECM producer cells are fibroblasts, keratinocytes, tenocytes, chondrocytes or any combination thereof.

6. An extracellular matrix according to claim 4, wherein the ECM producer cells are genetically modified for producing the LOX enzyme, or a fragment thereof, and/or BMP1 or a fragment thereof.

7. (canceled)

8. (canceled)

9. (canceled)

10. (canceled)

11. (canceled)

12. (canceled)

13. (canceled)

14. (canceled)

15. An in vitro method for obtaining an extracellular matrix (ECM) according to claim 1 comprising incubating ECM producer cells in the presence of a composition comprising a lysyl oxidase (LOX), or a fragment thereof, and bone morphogenetic protein-1 (BMP1), or a fragment thereof.

16. The method according to claim 15, wherein LOX protein comprises an amino acid sequence with an identity of at least 70, 75, 80, 85, 90, 95, 96, 97, 98, 99% with SEQ ID NO: 1.

17. The method according to claim 15, wherein BMP1 protein comprises an amino acid sequence with an identity of at least 70, 75, 80, 85, 90, 95, 96, 97, 98, 99% with SEQ ID NO: 2.

18. The method according to claim 15, wherein the ECM producer cells are fibroblasts, keratinocytes, tenocytes, chondrocytes or any combination thereof.

19. The method according to claim 15, wherein the ECM producer cells are genetically modified for producing the LOX enzyme, or a fragment thereof, and/or BMP1 or a fragment thereof.

20. An in vitro method for regulating the differentiation of mesenchymal stem cells comprising culturing the mesenchymal stem cells (ECM) in an extracellular matrix according to claim 1, wherein the ECM is decellularized.

21. A method for increasing the deposit of collagen in an extracellular matrix (ECM) comprising cultivating ECM producer cells in the presence of a composition comprising a lysyl oxidase (LOX) and bone morphogenetic protein-1 (BMP1), or culturing ECM producer cells genetically modified for producing the LOX enzyme and/or BMP 1.

22. The method according to claim 21, wherein LOX protein comprises an amino acid sequence with an identity of at least 70, 75, 80, 85, 90, 95, 96, 97, 98, 99% with SEQ ID NO: 1.

23. The method according to claim 21, wherein BMP1 protein comprises an amino acid sequence with an identity of at least 70, 75, 80, 85, 90, 95, 96, 97, 98, 99% with SEQ ID NO: 2.

24. The method according to claim 21, wherein the ECM producer cells are fibroblasts, keratinocytes, tenocytes, chondrocytes or any combination thereof.


SEQUENCE LISTING (11 sequences)

SEQ ID NO: 1 - protein, 417 aa, Homo sapiens
MRFAWTVLLLGPLQLCALVHCAPPAAGQQQPPREPPAAPGAWRQQIQWENNGQVFSLLSLGSQYQPQRRRDPGAAVPGAANASAQQPRTPILLIRDNRTAAARTRTAGSSGVTAGRPRPTARHWFQAGYSTSRAREAGASRAENQTAPGEVPALSNLRPPSRVDGMVGDDPYNPYKYSDDNPYYNYYDTYERPRPGGRYRPGYGTGYFQYGLPDLVADPYYIQASTYVQKMSMYNLRCAAEENCLASTAYRADVRDYDHRVLLRFPQRVKNQGTSDFLPSRPRYSWEWHSCHQHYHSMDEFSHYDLLDANTQRRVAEGHKASFCLEDTSCDYGYHRRFACTAHTQGLSPGCYDTYGADIDCQWIDITDVKPGNYILKVSVNPSYLVPESDYTNNVVRCDIRYTGHHAYASGCTISPY

SEQ ID NO: 2 - protein, 986 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSDKDECSKDNGGCQQDCVNTFGSYECQCRSGFVLHDNKHDCKEAGCDHKVTSTSGTITSPNWPDKYPSKKECTWAISSTPGHRVKLTFMEMDIESQPECAYDHLEVFDGRDAKAPVLGRFCGSKKPEPVLATGSRMFLRFYSDNSVQRKGFQASHATECGGQVRADVKTKDLYSHAQFGDNNYPGGVDCEWVIVAEEGYGVELVFQTFEVEEETDCGYDYMELFDGYDSTAPRLGRYCGSGPPEEVYSAGDSVLVKFHSDDTITKKGFHLRYTSTKFQDTLHSRK

SEQ ID NO: 3 - protein, 730 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSEKRPALQPPRGRPHQLKFRVQKRNRTPQ

SEQ ID NO: 4 - protein, 717 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSGGELFGLLGHPPRRP

SEQ ID NO: 5 - protein, 622 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEGCYDLQVGKPLLWDRHCFRLSTHGPEMLGTALRG

SEQ ID NO: 6 - protein, 813 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSGGLNGDQQPLAQPLSADPPGPGDLPFFVNHSALHPGSWRGQGTDTHIYQAWSYCSAPMPWSTLCPPHPQPCTETHTPTRTHMCTHIAPSHKKPAEDPHWGHRGSALRARGI

SEQ ID NO: 7 - protein, 964 aa, Homo sapiens
MRGGQSQSVFWGDIALDDEDLNIFQIDRTIDLTQNPFGNLGHTTGGLGDHAMSKKRGALYQLIDRIRRIGFGLEQNNTVKGKVPLQFSGQNEKNRVPRAATSRTERIWPGGVIPYVIGGNFTGSQRAMFKQAMRHWEKHTCVTFIERSDEESYIVFTYRPCGCCSYVGRRGNGPQAISIGKNCDKFGIVVHELGHVIGFWHEHTRPDRDNHVTIIRENIQPGQEYNFLKMEPGEVNSLGERYDFDSIMHYARNTFSRGMFLDTILPSRDDNGIRPAIGQRTRLSKGDIAQARKLYRCPACGETLQESNGNLSSPGFPNGYPSYTHCIWRVSVTPGEKIVLNFTTMDLYKSSLCWYDYIEVRDGYWRKSPLLGRFCGDKLPEVLTSTDSRMWIEFRSSSNWVGKGFAAVYEAICGGEIRKNEGQIQSPNYPDDYRPMKECVWKITVSESYHVGLTFQSFEIERHDNCAYDYLEVRDGTSENSPLIGRFCGYDKPEDIRSTSNTLWMKFVSDGTVNKAGFAANFFKEEDECAKPDRGGCEQRCLNTLGSYQCACEPGYELGPDRRSCEAACGGLLTKLNGTITTPGWPKEYPPNKNCVWQVVAPTQYRISVKFEFFELEGNEVCKYDYVEIWSGLSSESKLHGKFCGAEVPEVITSQFNNMRIEFKSDNTVSKKGFKAHFFSDKDECSKDNGGCQHECVNTMGSYMCQCRNGFVLHDNKHDCKEAECEQKIHSPSGLITSPNWPDKYPSRKECTWEISATPGHRIKLAFSEFEIEQHQECAYDHLEVFDGETEKSPILGRLCGNKIPDPLVATGNKMFVRFVSDASVQRKGFQATHSTECGGRLKAESKPRDLYSHAQFGDNNYPGQVDCEWLLVSERGSRLELSFQTFEVEEEADCGYDYVELFDGLDSTAVGLGRFCGSGPPEEIYSIGDSVLIHFHTDDTINKKGFHIRYKSIRYPDTTHTKK

SEQ ID NO: 8 - protein, 837 aa, Homo sapiens
MFKQAMRHWEKHTCVTFIERSDEESYIVFTYRPCGCCSYVGRRGNGPQAISIGKNCDKFGIVVHELGHVIGFWHEHTRPDRDNHVTIIRENIQPGQEYNFLKMEPGEVNSLGERYDFDSIMHYARNTFSRGMFLDTILPSRDDNGIRPAIGQRTRLSKGDIAQARKLYRCPACGETLQESNGNLSSPGFPNGYPSYTHCIWRVSVTPGEKIVLNFTTMDLYKSSLCWYDYIEVRDGYWRKSPLLGRFCGDKLPEVLTSTDSRMWIEFRSSSNWVGKGFAAVYEAICGGEIRKNEGQIQSPNYPDDYRPMKECVWKITVSESYHVGLTFQSFEIERHDNCAYDYLEVRDGTSENSPLIGRFCGYDKPEDIRSTSNTLWMKFVSDGTVNKAGFAANFFKEEDECAKPDRGGCEQRCLNTLGSYQCACEPGYELGPDRRSCEAACGGLLTKLNGTITTPGWPKEYPPNKNCVWQVVAPTQYRISVKFEFFELEGNEVCKYDYVEIWSGLSSESKLHGKFCGAEVPEVITSQFNNMRIEFKSDNTVSKKGFKAHFFSDKDECSKDNGGCQHECVNTMGSYMCQCRNGFVLHDNKHDCKEAECEQKIHSPSGLITSPNWPDKYPSRKECTWEISATPGHRIKLAFSEFEIEQHQECAYDHLEVFDGETEKSPILGRLCGNKIPDPLVATGNKMFVRFVSDASVQRKGFQATHSTECGGRLKAESKPRDLYSHAQFGDNNYPGQVDCEWLLVSERGSRLELSFQTFEVEEEADCGYDYVELFDGLDSTAVGLGRFCGSGPPEEIYSIGDSVLIHFHTDDTINKKGFHIRYKSIRYPDTTHTKK

SEQ ID NO: 9 - protein, 392 aa, Homo sapiens
MGLGTLSPRMLVWLVASGIVFYGELWVCAGLDYDYTFDGNEEDKTETIDYKDPCKAAVFWGDIALDDEDLNIFQIDRTIDLTQNPFGNLGHTTGGLGDHAMSKKRGALYQLIDRIRRIGFGLEQNNTVKGKVPLQFSGQNEKNRVPRAATSRTERIWPGGVIPYVIGGNFTGSQRAMFKQAMRHWEKHTCVTFIERSDEESYIVFTYRPCGCCSYVGRRGNGPQAISIGKNCDKFGIVVHELGHVIGFWHEHTRPDRDNHVTIIRENIQPGQEYNFLKMEPGEVNSLGERYDFDSIMHYARNTFSRGMFLDTILPSRDDNGIRPAIGQRTRLSKGDIAQARKLYRCPACGETLQESNGNLSSPGFPNGYPSYTHCIWRVSVTPGEKVVFSLC

SEQ ID NO: 10 - protein, 1013 aa, Homo sapiens
MGLGTLSPRMLVWLVASGIVFYGELWVCAGLDYDYTFDGNEEDKTETIDYKDPCKAAVFWGDIALDDEDLNIFQIDRTIDLTQNPFGNLGHTTGGLGDHAMSKKRGALYQLIDRIRRIGFGLEQNNTVKGKVPLQFSGQNEKNRVPRAATSRTERIWPGGVIPYVIGGNFTGSQRAMFKQAMRHWEKHTCVTFIERSDEESYIVFTYRPCGCCSYVGRRGNGPQAISIGKNCDKFGIVVHELGHVIGFWHEHTRPDRDNHVTIIRENIQPGQEYNFLKMEPGEVNSLGERYDFDSIMHYARNTFSRGMFLDTILPSRDDNGIRPAIGQRTRLSKGDIAQARKLYRCPACGETLQESNGNLSSPGFPNGYPSYTHCIWRVSVTPGEKIVLNFTTMDLYKSSLCWYDYIEVRDGYWRKSPLLGRFCGDKLPEVLTSTDSRMWIEFRSSSNWVGKGFAAVYEAICGGEIRKNEGQIQSPNYPDDYRPMKECVWKITVSESYHVGLTFQSFEIERHDNCAYDYLEVRDGTSENSPLIGRFCGYDKPEDIRSTSNTLWMKFVSDGTVNKAGFAANFFKEEDECAKPDRGGCEQRCLNTLGSYQCACEPGYELGPDRRSCEAACGGLLTKLNGTITTPGWPKEYPPNKNCVWQVVAPTQYRISVKFEFFELEGNEVCKYDYVEIWSGLSSESKLHGKFCGAEVPEVITSQFNNMRIEFKSDNTVSKKGFKAHFFSDKDECSKDNGGCQHECVNTMGSYMCQCRNGFVLHDNKHDCKEAECEQKIHSPSGLITSPNWPDKYPSRKECTWEISATPGHRIKLAFSEFEIEQHQECAYDHLEVFDGETEKSPILGRLCGNKIPDPLVATGNKMFVRFVSDASVQRKGFQATHSTECGGRLKAESKPRDLYSHAQFGDNNYPGQVDCEWLLVSERGSRLELSFQTFEVEEEADCGYDYVELFDGLDSTAVGLGRFCGSGPPEEIYSIGDSVLIHFHTDDTINKKGFHIRYKSIRYPDTTHTKK

SEQ ID NO: 11 - protein, 1015 aa, Homo sapiens
MPRATALGALVSLLLLLPLPRGAGGLGERPDATADYSELDGEEGTEQQLEHYHDPCKAAVFWGDIALDEDDLKLFHIDKARDWTKQTVGATGHSTGGLEEQASESSPDTTAMDTGTKEAGKDGRENTTLLHSPGTLHAAAKTFSPRVRRATTSRTERIWPGGVIPYVIGGNFTGSQRAIFKQAMRHWEKHTCVTFIERTDEESFIVFSYRTCGCCSYVGRRGGGPQAISIGKNCDKFGIVAHELGHVVGFWHEHTRPDRDQHVTIIRENIQPGQEYNFLKMEAGEVSSLGETYDFDSIMHYARNTFSRGVFLDTILPRQDDNGVRPTIGQRVRLSQGDIAQARKLYKCPACGETLQDTTGNFSAPGFPNGYPSYSHCVWRISVTPGEKIVLNFTSMDLFKSRLCWYDYVEVRDGYWRKAPLLGRFCGDKIPEPLVSTDSRLWVEFRSSSNILGKGFFAAYEATCGGDMNKDAGQIQSPNYPDDYRPSKECVWRITVSEGFHVGLTFQAFEIERHDSCAYDYLEVRDGPTEESALIGHFCGYEKPEDVKSSSNRLWMKFVSDGSINKAGFAANFFKEVDECSWPDHGGCEHRCVNTLGSYKCACDPGYELAADKKMCEVACGGFITKLNGTITSPGWPKEYPTNKNCVWQVVAPAQYRISLQFEVFELEGNDVCKYDFVEVRSGLSPDAKLHGRFCGSETPEVITSQSNNMRVEFKSDNTVSKRGFRAHFFSDKDECAKDNGGCQHECVNTFGSYLCRCRNGYWLHENGHDCKEAGCAHKISSVEGTLASPNWPDKYPSRRECTWNISSTAGHRVKLTFNEFEIEQHQECAYDHLEMYDGPDSLAPILGRFCGSKKPDPTVASGSSMFLRFYSDASVQRKGFQAVHSTECGGRLKAEVQTKELYSHAQFGDNNYPSEARCDWVIVAEDGYGVELTFRTFEVEEEADCGYDYMEAYDGYDSSAPRLGRFCGSGPLEEIYSAGDSLMIRFRTDDTINKKGFHARYTSTKFQDALHMKK